(19) United States
(12) Patent Application Publication
(10) Pub. No.: US 2023/0245305 A1
(43) Pub. Date: Aug. 3, 2023

(54) IMAGE-BASED VARIANT PATHOGENICITY DETERMINATION

(71) Applicants: Illumina, Inc., San Diego, CA (US); Illumina Cambridge Limited, Cambridge (GB)

(72) Inventors: Tobias Hamp, Cambridge (GB); Hong Gao, Palo Alto, CA (US); Kai-How Farh, Hillsborough, CA (US)

(21) Appl. No.: 18/160,855

(22) Filed: Jan. 27, 2023

Related U.S. Application Data
(60) Provisional application No. 63/304,544, filed on Jan. 28, 2022.

(51) Int. Cl. G06T 7/00; G06T 7/70; G06T 17/00

(52) U.S. Cl. CPC G06T 7/0012; G06T 7/70; G06T 17/00; G06T 2207/20084

(57) ABSTRACT

Described herein are technologies for classifying a protein structure (such as technologies for classifying the pathogenicity of a protein structure related to a nucleotide variant). Such a classification is based on two-dimensional images taken from a three-dimensional image of the protein structure. With respect to some implementations, described herein are multi-view convolutional neural networks (CNNs) for classifying a protein structure based on inputs of two-dimensional images taken from a three-dimensional image of the protein structure. In some implementations, a computer-implemented method of determining pathogenicity of variants includes accessing a structural rendition of amino acids, capturing images of those parts of the structural rendition that contain a target amino acid from the amino acids, and, based on the images, determining pathogenicity of a nucleotide variant that mutates the target amino acid into an alternate amino acid.

### CROSS-REFERENCE TO RELATED APPLICATIONS

This application claims the benefit of, and priority to, U.S. Provisional Application No. 63/304,544, entitled “IMAGE-BASED VARIANT PATHOGENICITY DETERMINATION,” filed Jan. 28, 2022. The aforementioned application is hereby incorporated by reference in its entirety.

### FIELD OF THE TECHNOLOGY DISCLOSED

The technology disclosed relates to artificial intelligence type computers and digital data processing systems and corresponding data processing methods and products for emulation of intelligence (i.e., knowledge based systems, reasoning systems, and knowledge acquisition systems); and including systems for reasoning with uncertainty (e.g., fuzzy logic systems), adaptive systems, machine learning systems, and artificial neural networks. In particular, the technology disclosed relates to using techniques for converting context of an artificial neural network (ANN) or another type of computing system that is trainable through machine learning.

Additionally, the technology disclosed relates to pre-processing of inputs for artificial intelligence type computers and digital data processing systems and corresponding data processing methods and products for emulation of intelligence as well as the actual pre-processed inputs themselves.

### INCORPORATIONS

The following are incorporated by reference for all purposes as if fully set forth herein:

U.S. Provisional Patent Application No. 63/253,122, titled “PROTEIN STRUCTURE-BASED PROTEIN LANGUAGE MODELS,” filed Oct. 6, 2021 (Attorney Docket No. ILLM 1050-1/IP-2164-PRV);

U.S. Provisional Patent Application No. 63/281,579, titled “PREDICTING VARIANT PATHOGENICITY FROM EVOLUTIONARY CONSERVATION USING THREE-DIMENSIONAL (3D) PROTEIN STRUCTURE VOXELS,” filed Nov. 19, 2021 (Attorney Docket No. ILLM 1060-1/IP-2270-PRV);

U.S. Provisional Patent Application No. 63/281,592, titled “COMBINED AND TRANSFER LEARNING OF A VARIANT PATHOGENICITY PREDICTOR USING GAPED AND NON-GAPED PROTEIN SAMPLES,” filed Nov. 19, 2021 (Attorney Docket No. ILLM 1061-1/IP-2271-PRV);

U.S. Patent Application No. 62/573,144, titled “TRAINING A DEEP PATHOGENICITY CLASSIFIER USING LARGE-SCALE BENIGN TRAINING DATA,” filed Oct. 16, 2017 (Attorney Docket No. ILLM 1000-1/IP-1611-PRV);

U.S. Patent Application No. 62/573,149, titled “PATHOGENICITY CLASSIFIER BASED ON DEEP CONVOLUTIONAL NEURAL NETWORKS (CNNs),” filed Oct. 16, 2017 (Attorney Docket No. ILLM 1000-2/IP-1612-PRV);

U.S. Patent Application No. 62/573,153, titled “DEEP SEMI-SUPERVISED LEARNING THAT GENERATES LARGE-SCALE PATHOGENIC TRAINING DATA,” filed Oct. 16, 2017 (Attorney Docket No. ILLM 1000-3/IP-1613-PRV);

U.S. Patent Application No. 62/582,898, titled “PATHOGENICITY CLASSIFICATION OF GENOMIC DATA USING DEEP CONVOLUTIONAL NEURAL NETWORKS (CNNs),” filed Nov. 7, 2017 (Attorney Docket No. ILLM 1000-4/IP-1618-PRV);

U.S. patent application Ser. No. 16/160,903, titled “DEEP LEARNING-BASED TECHNIQUES FOR TRAINING DEEP CONVOLUTIONAL NEURAL NETWORKS,” filed on Oct. 15, 2018 (Attorney Docket No. ILLM 1000-5/IP-1611-US);

U.S. patent application Ser. No. 16/160,986, titled “DEEP CONVOLUTIONAL NEURAL NETWORKS FOR VARIANT CLASSIFICATION,” filed on Oct. 15, 2018 (Attorney Docket No. ILLM 1000-6/IP-1612-US);

U.S. patent application Ser. No. 16/160,968, titled “SEMI-SUPERVISED LEARNING FOR TRAINING AN ENSEMBLE OF DEEP CONVOLUTIONAL NEURAL NETWORKS,” filed on Oct. 15, 2018 (Attorney Docket No. ILLM 1000-7/IP-1613-US);

U.S. patent application Ser. No. 16/407,149, titled “DEEP LEARNING-BASED TECHNIQUES FOR PRE-TRAINING DEEP CONVOLUTIONAL NEURAL NETWORKS,” filed May 8, 2019 (Attorney Docket No. ILLM 1010-1/IP-1734-US);

U.S. patent application Ser. No. 17/232,056, titled “DEEP CONVOLUTIONAL NEURAL NETWORKS TO PREDICT VARIANT PATHOGENICITY USING THREE-DIMENSIONAL (3D) PROTEIN STRUCTURES,” filed on Apr. 15, 2021, (Atty. Docket No. ILLM 1037-2/IP-2051-US);

U.S. Patent Application No. 63/175,495, titled “MULTI-CHANNEL PROTEIN VOXELIZATION TO PREDICT VARIANT PATHOGENICITY USING DEEP CONVOLUTIONAL NEURAL NETWORKS,” filed on Apr. 15, 2021, (Atty. Docket No. ILLM 1047-1/IP-2142-PRV);

Sundaram, L. et al. Predicting the clinical impact of human mutation with deep neural networks. Nat. Genet. 50, 1161-1170 (2018);

Jaganathan, K. et al. Predicting splicing from primary sequence with deep learning. Cell 176, 535-548 (2019);

U.S. Patent Application No. 63/175,767, titled “EFFICIENT VOXELIZATION FOR DEEP LEARNING,” filed on Apr. 16, 2021, (Atty. Docket No. ILLM 1048-1/IP-2143-PRV); and

U.S. patent application Ser. No. 17/468,411, titled “ARTIFICIAL INTELLIGENCE-BASED ANALYSIS OF PROTEIN THREE-DIMENSIONAL (3D) STRUCTURES,” filed on Sep. 7, 2021, (Atty. Docket No. ILLM 1037-3/IP-2051A-US).

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Genomics, in the broad sense, also referred to as functional genomics, aims to characterize the function of every genomic element of an organism by using genome-scale assays such as genome sequencing, transcriptome profiling and proteomics. Genomics arose as a data-driven science—it operates by discovering novel properties from explorations of genome-scale data rather than by testing preconceived models and hypotheses. Applications of genomics include finding associations between genotype and phenotype, discovering biomarkers for patient stratification, predicting the function of genes, and charting biochemically active genomic regions such as transcriptional enhancers.

Genomics data are too large and too complex to be mined solely by visual investigation of pairwise correlations. Instead, analytical tools are required to support the discovery of unanticipated relationships, to derive novel hypotheses and models and to make predictions. Unlike some algorithms, in which assumptions and domain expertise are hard coded, machine learning algorithms are designed to automatically detect patterns in data. Hence, machine learning algorithms are suited to data-driven sciences and, in particular, to genomics. However, the performance of machine learning algorithms can strongly depend on how the data are represented, that is, on how each variable (also called a feature) is computed. For instance, to classify a tumor as malign or benign from a fluorescent microscopy image, a preprocessing algorithm could detect cells, identify the cell type, and generate a list of cell counts for each cell type.

A machine learning model can take the estimated cell counts, which are examples of handcrafted features, as input features to classify the tumor. A central issue is that classification performance depends heavily on the quality and the relevance of these features. For example, relevant visual features such as cell morphology, distances between cells or localization within an organ are not captured in cell counts, and this incomplete representation of the data may reduce classification accuracy.

Deep learning, a subdiscipline of machine learning, addresses this issue by embedding the computation of features into the machine learning model itself to yield end-to-end models. This outcome has been realized through the development of deep neural networks, machine learning models that comprise successive elementary operations, which compute increasingly more complex features by taking the results of preceding operations as input. Deep neural networks are able to improve prediction accuracy by discovering relevant features of high complexity, such as the cell morphology and spatial organization of cells in the above example. The construction and training of deep neural networks have been enabled by the explosion of data, algorithmic advances, and substantial increases in computational capacity, particularly through the use of graphical processing units (GPUs).

The goal of supervised learning is to obtain a model that takes features as input and returns a prediction for a so-called target variable. An example of a supervised learning problem is one that predicts whether an intron is spliced out or not (the target) given features on the RNA such as the presence or absence of the canonical splice site sequence, the location of the splicing branchpoint or intron length. Training a machine learning model refers to learning its parameters, which commonly involves minimizing a loss function on training data with the aim of making accurate predictions on unseen data.

For many supervised learning problems in computational biology, the input data can be represented as a table with multiple columns, or features, each of which contains numerical or categorical data that are potentially useful for making predictions. Some input data are naturally represented as features in a table (such as temperature or time), whereas other input data need to be first transformed (such as deoxyribonucleic acid (DNA) sequence into k-mer counts) using a process called feature extraction to fit a tabular representation. For the intron-splicing prediction problem, the presence or absence of the canonical splice site sequence, the location of the splicing branchpoint and the intron length can be preprocessed features collected in a tabular format. Tabular data are standard for a wide range of supervised machine learning models, ranging from simple linear models, such as logistic regression, to more flexible nonlinear models, such as neural networks and many others.

Logistic regression is a binary classifier, that is, a supervised learning model that predicts a binary target variable. Specifically, logistic regression predicts the probability of the positive class by computing a weighted sum of the input features mapped to the [0,1] interval using the sigmoid function, a type of activation function. The parameters of logistic regression, or other linear classifiers that use different activation functions, are the weights in the weighted sum. Linear classifiers fail when the classes, for instance, that of an intron spliced out or not, cannot be well discriminated with a weighted sum of input features. To improve predictive performance, new input features can be manually added by transforming or combining existing features in new ways, for example, by taking powers or pairwise products.

Neural networks use hidden layers to learn these nonlinear feature transformations automatically. Each hidden layer can be thought of as multiple linear models with their output transformed by a nonlinear activation function, such as the sigmoid function or the more popular rectified-linear unit (ReLU). Together, these layers compose the input features into relevant complex patterns, which facilitates the task of distinguishing two classes.

Deep neural networks use many hidden layers, and a layer is said to be fully-connected when each neuron receives inputs from all neurons of the preceding layer. Neural networks are commonly trained using stochastic gradient descent, an algorithm suited to training models on very large data sets. Implementation of neural networks using modern deep learning frameworks enables rapid prototyping with different architectures and data sets. Fully-connected neural networks can be used for a number of genomics applications, which include predicting the percentage of exons spliced in for a given sequence from sequence features such as the presence of binding motifs of splice factors or sequence conservation; prioritizing potential disease-causing genetic variants; and predicting cis-regulatory elements in a given genomic region using features such as chromatin marks, gene expression and evolutionary conservation.

Local dependencies in spatial and longitudinal data must be considered for effective predictions. For example, shuffling a DNA sequence or the pixels of an image severely disrupts informative patterns. These local dependencies set spatial or longitudinal data apart from tabular data, for which the ordering of the features is arbitrary. Consider the problem of classifying genomic regions as bound versus unbound by a particular transcription factor, in which bound regions are defined as high-confidence binding events in chromatin immunoprecipitation following by sequencing (ChIP-seq) data. Transcription factors bind to DNA by recognizing sequence motifs. A fully-connected layer based on sequence-derived features, such as the number of k-mer instances or the position weight matrix (PWM) matches in the sequence, can be used for this task. As k-mer or PWM instance frequencies are robust to shifting motifs within the sequence, such models could generalize well to sequences with the same motifs located at different positions. However, they would fail to recognize patterns in which transcription factor binding depends on a combination of multiple motifs with well-defined spacing. Furthermore, the number of possible k-mers increases exponentially with k-mer length, which poses both storage and overfitting challenges.

A convolutional layer is a special form of fully-connected layer in which the same fully-connected layer is applied locally, for example, in a 6 bp window, to all sequence positions. This approach can also be viewed as scanning the sequence using multiple PWMs, for example, for transcription factors GATA1 and TAL1. By using the same model parameters across positions, the total number of parameters is drastically reduced, and the network is able to detect a motif at positions not seen during training. Each convolutional layer scans the sequence with several filters by producing a scalar value at every position, which quantifies the match between the filter and the sequence. As in fully-connected neural networks, a nonlinear activation function (commonly ReLU) is applied at each layer. Next, a pooling operation is applied, which aggregates the activations in contiguous bins across the positional axis, commonly taking the maximal or average activation for each channel. Pooling reduces the effective sequence length and coarsens the signal. The subsequent convolutional layer composes the output of the previous layer and is able to detect whether a GATA1 motif and TAL1 motif were present at some distance range. Finally, the output of the convolutional layers can be used as input to a fully-connected neural network to perform the final prediction task. Hence, different types of neural network layers (e.g., fully-connected layers and convolutional layers) can be combined within a single neural network.

Convolutional neural networks (CNNs) can predict various molecular phenotypes on the basis of DNA sequence alone. Applications include classifying transcription factor binding sites and predicting molecular phenotypes such as chromatin features, DNA contact maps, DNA methylation, gene expression, translation efficiency, RBP binding, and microRNA (miRNA) targets. In addition to predicting molecular phenotypes from the sequence, convolutional neural networks can be applied to more technical tasks traditionally addressed by handcrafted bioinformatics pipelines. For example, convolutional neural networks can predict the specificity of guide RNA, denoise ChIP-seq, enhance Hi-C data resolution, predict the laboratory of origin from DNA sequences and call genetic variants. Convolutional neural networks have also been employed to model long-range dependencies in the genome. Although interacting regulatory elements may be distantly located on the unfolded linear DNA sequence, these elements are often proximal in the actual 3D chromatin conformation. Hence, modelling molecular phenotypes from the linear DNA sequence, albeit a crude approximation of the chromatin, can be improved by allowing for long-range dependencies and allowing the model to implicitly learn aspects of the 3D organization, such as promoter-enhancer looping. This is achieved by using dilated convolutions, which have a receptive field of up to 32 kb. Dilated convolutions also allow splice sites to be predicted from sequence using a receptive field of 10 kb, thereby enabling the integration of genetic sequence across distances as long as typical human introns (See Jaganathan, K. et al. Predicting splicing from primary sequence with deep learning. Cell 176, 535-548 (2019)).

Different types of neural network can be characterized by their parameter-sharing schemes. For example, fully-connected layers have no parameter sharing, whereas convolutional layers impose translational invariance by applying the same filters at every position of their input. Recurrent neural networks (RNNs) are an alternative to convolutional neural networks for processing sequential data, such as DNA sequences or time series, that implement a different parameter-sharing scheme. Recurrent neural networks apply the same operation to each sequence element. The operation takes as input the memory of the previous sequence element and the new input. It updates the memory and optionally emits an output, which is either passed on to subsequent layers or is directly used as model predictions. By applying the same model at each sequence element, recurrent neural networks are invariant to the position index in the processed sequence. For example, a recurrent neural network can detect an open reading frame in a DNA sequence regardless of the position in the sequence. This task requires the recognition of a certain series of inputs, such as the start codon followed by an in-frame stop codon.

The main advantage of recurrent neural networks over convolutional neural networks is that they are, in theory, able to carry over information through infinitely long sequences via memory. Furthermore, recurrent neural networks can naturally process sequences of widely varying length, such as mRNA sequences. However, convolutional neural networks combined with various tricks (such as dilated convolutions) can reach comparable or even better performances than recurrent neural networks on sequence-modelling tasks, such as audio synthesis and machine translation. Recurrent neural networks can aggregate the outputs of convolutional neural networks for predicting single-cell DNA methylation states, RBP binding, transcription factor binding, and DNA accessibility. Moreover, because recurrent neural networks apply a sequential operation, they cannot be easily parallelized and are hence much slower to compute than convolutional neural networks.

Each human has a unique genetic code, though a large portion of the human genetic code is common for all humans. In some cases, a human genetic code may include an outlier, called a genetic variant, that may be common among individuals of a relatively small group of the human population. For example, a particular human protein may comprise a specific sequence of amino acids, whereas a variant of that protein may differ by one amino acid in the otherwise same specific sequence.

Genetic variants may be pathogenetic, leading to diseases. Though most of such genetic variants have been depleted from genomes by natural selection, an ability to identify which genetic variants are likely to be pathogenic can help researchers focus on these genetic variants to gain an understanding of the corresponding diseases and their diagnostics, treatments, or cures. The clinical interpretation of millions of human genetic variants remains unclear. Some of the most frequent pathogenic variants are single nucleotide missense mutations that change the amino acid of a protein. However, not all missense mutations are pathogenic.

Models that can predict molecular phenotypes directly from biological sequences can be used as in silico perturbation tools to probe the associations between genetic variation and phenotypic variation and have emerged as new methods for quantitative trait loci identification and variant prioritization. These approaches are of major importance given that the majority of variants identified by genome-wide association studies of complex phenotypes are non-coding, which makes it challenging to estimate their effects and contribution to phenotypes. Moreover, linkage disequilibrium results in blocks of variants being co-inherited, which creates difficulties in pinpointing individual causal variants. Thus, sequence-based deep learning models that can be used as interrogation tools for assessing the impact of such variants offer a promising approach to find potential drivers of complex phenotypes. One example includes predicting the effect of non-coding single-nucleotide variants and short insertions or deletions (indels) indirectly from the difference between two variants in terms of transcription factor binding, chromatin accessibility or gene expression predictions. Another example includes predicting novel splice site creation from sequence or quantitative effects of genetic variants on splicing.

End-to-end deep learning approaches for variant effect predictions are applied to predict the pathogenicity of missense variants from protein sequence and sequence conservation data (See Sundaram, L. et al. Predicting the clinical impact of human mutation with deep neural networks. Nat. Genet. 50, 1161-1170 (2018), referred to herein as “PrimateAI”). PrimateAI uses deep neural networks trained on variants of known pathogenicity with data augmentation using cross-species information. In particular, PrimateAI uses sequences of wild-type and mutant proteins to compare the difference and decide the pathogenicity of mutations using the trained deep neural networks. Such an approach which utilizes the protein sequences for pathogenicity prediction is promising because it can avoid the circularity problem and overfitting to previous knowledge. However, compared to the adequate number of data to train the deep neural networks effectively, the number of clinical data available in ClinVar is relatively small. To overcome this data scarcity, PrimateAI uses common human variants and variants from primates as benign data while simulated variants based on trinucleotide context were used as unlabeled data.

PrimateAI outperforms prior methods when trained directly upon sequence alignments. PrimateAI learns important protein domains, conserved amino acid positions, and sequence dependencies directly from the training data consisting of about 120,000 human samples. PrimateAI substantially exceeds the performance of other variant pathogenicity prediction tools in differentiating benign and pathogenic de-novo mutations in candidate developmental disorder genes, and in reproducing prior knowledge in ClinVar. These results suggest that PrimateAI is an important step forward for variant classification tools that may lessen the reliance of clinical reporting on prior knowledge.

Central to protein biology is the understanding of how structural elements give rise to observed function. The surfeit of protein structural data enables development of computational methods to systematically derive rules governing structural-functional relationships. However, performance of these methods depends critically on the choice of protein structural representation.

Protein sites are microenvironments within a protein structure, distinguished by their structural or functional role. A site can be defined by a three-dimensional (3D) location and a local neighborhood around this location in which the structure or function exists. Central to rational protein engineering is the understanding of how the structural arrangement of amino acids creates functional characteristics within protein sites. Determination of the structural and functional roles of individual amino acids within a protein provides information to help engineer and alter protein functions. Identifying functionally or structurally important amino acids allows focused engineering efforts such as site-directed mutagenesis for altering targeted protein functional properties. Alternatively, this knowledge can help avoid engineering designs that would abolish a desired function.

Since it has been established that structure is far more conserved than sequence, the increase in protein structural data provides an opportunity to systematically study the underlying pattern governing the structural-functional relationships using data-driven approaches. A fundamental aspect of any computational protein analysis is how protein structural information is represented. The performance of machine learning methods often depends more on the choice of data representation than the machine learning algorithm employed. Good representations efficiently capture the most critical information while poor representations create a noisy distribution with no underlying patterns.

The surfeit of protein structures and the recent success of deep learning algorithms provide an opportunity to develop tools for automatically extracting task specific representations of protein structures. Therefore, an opportunity arises to predict variant pathogenicity using multi-channel voxelized representations of 3D protein structures as input to deep neural networks.

### SUMMARY

With respect to some implementations, described herein are multi-view convolutional neural networks (CNNs) for classifying protein structures. Also, with respect to some implementations, described herein are processed inputs for such multi-view CNNs. In providing such implementations and others, the systems and methods described herein overcome some technical problems in classifying protein structures via artificial intelligence type computers and digital data processing systems and corresponding data processing methods and products for emulation of intelligence. Also, the technologies disclosed herein provide specific technical solutions to at least overcome the technical problems mentioned herein as well as other technical problems not described herein but recognized by those skilled in the art.

With respect to some implementations, disclosed herein are computerized methods for classifying protein structures via trainable computing systems and computerized methods for pre-processing inputs for the trainable computing systems, as well as a non-transitory computer-readable storage medium for carrying out technical operations of the computerized methods. The non-transitory computer-readable storage medium has tangibly stored thereon, or tangibly encoded thereon, computer readable instructions that when executed by one or more devices (e.g., one or more personal computers or servers) cause at least one processor to perform (1) a method for classifying protein structures via trainable computing systems, (2) a method for pre-processing inputs for the trainable computing systems, or (3) a method for performing a combination of the method for classifying protein structures and the method for pre-processing the inputs.

In some implementations, a computer-implemented method of determining pathogenicity of variants includes accessing a structural rendition of amino acids of a protein, capturing a plurality of images of those parts of the structural rendition that contain a target amino acid from the amino acids, and, based at least in part on the plurality of images, determining pathogenicity of a nucleotide variant that mutates the target amino acid into an alternate amino acid.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The color drawings also may be available in PAIR via the Supplemental Content tab.

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings.

FIG. 1 depicts one implementation of multi-view convolutional neural networks for 3D shape recognition.

FIG. 2 depicts a system, including a two-dimensional (2D) image classification network, that generates classifications of protein structures, in accordance with some implementations of the present disclosure.

FIGS. 3A, 3B, 3C and 3D depict 2D images of a protein structure generated from a three-dimensional (3D) image of the protein structure, in accordance with some implementations of the present disclosure.

FIGS. 4, 5, and 6 depict methods using a 2D image classification network to generate classifications of protein structures, in accordance with some implementations of the present disclosure.

FIG. 7 depicts a system, including convolutional neural networks, that generates classifications of protein structures, in accordance with some implementations of the present disclosure.

FIGS. 8 and 9 depict methods using convolutional neural networks (CNNs) to generate classifications of protein structures, in accordance with some implementations of the present disclosure.

FIGS. 10, 11, and 12 depict methods of determining pathogenicity of variants, in accordance with some implementations of the present disclosure.

FIG. 13 depicts a block diagram of example aspects of a computing system, in accordance with some implementations of the present disclosure.

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

### Multi-View Convolutional Neural Networks for 3D Shape Recognition

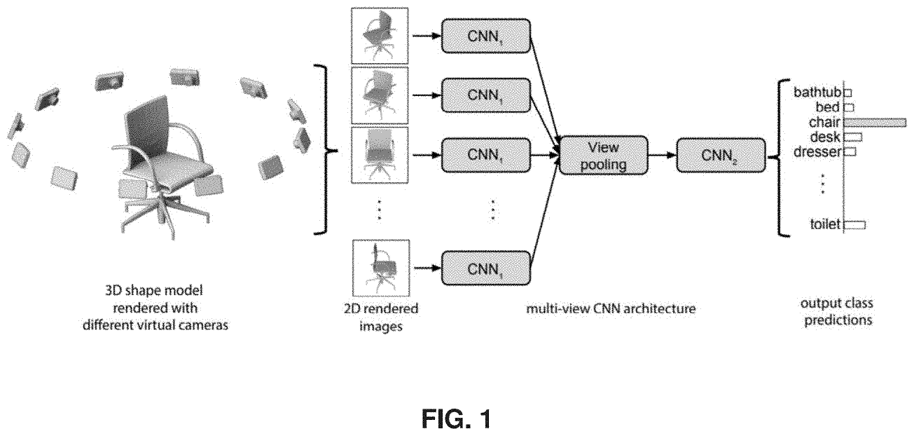

FIG. 1 shows one implementation of multi-view convolutional neural networks (otherwise known as multi-view CNNs) for three-dimensional (3D) shape recognition. FIG. 1 can be found in the article titled “Multi-view Convolutional Neural Networks for 3D Shape Recognition” by Hang Su et al., published May 5, 2015 for the 2015 IEEE International Conference on Computer Vision (ICCV) (Su, Hang et al. “Multi-view Convolutional Neural Networks for 3D Shape Recognition.” 2015 IEEE International Conference on Computer Vision (ICCV) (2015): 945-953.). FIG. 1 depicts a 3D shape being rendered from multiple different views and the views being passed through multiple corresponding first convolutional neural networks (CNNs) to extract view based features. The features are then pooled across views and passed through a second convolutional neural network (CNN) to obtain a compact shape descriptor.

### Genomics and Deep Learning

Genomics, in the broad sense, also referred to as functional genomics, aims to characterize the function of every genomic element of an organism by using genome-scale assays such as genome sequencing, transcriptome profiling and proteomics. Genomics arose as a data-driven science—it operates by discovering novel properties from explorations of genome-scale data rather than by testing preconceived models and hypotheses. Applications of genomics include finding associations between genotype and phenotype, discovering biomarkers for patient stratification, predicting the function of genes, and charting biochemically active genomic regions such as transcriptional enhancers.

Genomics data are too large and too complex to be mined solely by visual investigation of pairwise correlations. Instead, analytical tools are required to support the discovery of unanticipated relationships, to derive novel hypotheses and models and to make predictions. Unlike some models, in which assumptions and domain expertise are hard coded, machine learning models are designed to automatically detect patterns in data. Hence, machine learning models are suited to data-driven sciences and, in particular, to genomics. However, the performance of machine learning models can strongly depend on how the data are represented, that is, on how each variable (also called a feature) is computed. For instance, to classify a tumor as malign or benign from a fluorescent microscopy image, a preprocessing model could detect cells, identify the cell type, and generate a list of cell counts for each cell type.

A machine learning model can take the estimated cell counts, which are examples of handcrafted features, as input features to classify the tumor. A central issue is that classification performance depends heavily on the quality and the relevance of these features. For example, relevant visual features such as cell morphology, distances between cells or localization within an organ are not captured in cell counts, and this incomplete representation of the data may reduce classification accuracy.

Deep learning, a subdiscipline of machine learning, addresses this issue by embedding the computation of features into the machine learning model itself to yield end-to-end models. This outcome has been realized through the development of deep neural networks, machine learning models that include successive elementary operations, which compute increasingly more complex features by taking the results of preceding operations as input. Deep neural networks are able to improve prediction accuracy by discovering relevant features of high complexity, such as the cell morphology and spatial organization of cells in the above example. The construction and training of deep neural networks have been enabled by the explosion of data, modelic advances, and substantial increases in computational capacity, particularly through the use of graphical processing units (GPUs).

Described herein are technologies for classifying a protein structure (such as technologies for classifying the pathogenicity of a protein structure related to a nucleotide variant). Such a classification is based on two-dimensional images taken from a three-dimensional image of the protein structure. With respect to some implementations, described herein are multi-view convolutional neural networks (CNNs) for classifying a protein structure based on inputs of two-dimensional images taken from a three-dimensional image of the protein structure. In some implementations, a computer-implemented method of determining pathogenicity of variants includes accessing a structural rendition of amino acids of a protein, capturing a plurality of images of those parts of the structural rendition that contain a target amino acid from the amino acids, and, based at least in part on the plurality of images, determining pathogenicity of a nucleotide variant that mutates the target amino acid into an alternate amino acid. The parts are captured by zooming-in to the parts. In other implementations, the parts are captured by filtering out other parts.

The actions in Figures disclosed herein can be implemented at least partially with and/or by one or more processors configured to receive or retrieve information, process the information, store results, and transmit the results. Other implementations may perform the actions in different orders and/or with different, fewer, or additional actions than those illustrated in the Figures. Multiple actions can be combined in some implementations. For convenience, this figure is described with reference to the system that carries out a method. The system is not necessarily part of the method. The actions in Figures disclosed herein can be executed in parallel or in sequence.

In one implementation, a phenotyping logic (e.g., a pathogenicity classifier) is a multilayer perceptron (MLP). In another implementation, the phenotyping logic is a feedforward neural network. In yet another implementation, the phenotyping logic is a fully-connected neural network. In a further implementation, the phenotyping logic is a fully convolution neural network. In yet further implementation, the phenotyping logic is a semantic segmentation neural network. In yet another further implementation, the phenotyping logic is a generative adversarial network (GAN) (e.g., CycleGAN, StyleGAN, pixelRNN, text-2-image, DiscoGAN, IsGAN).

In one implementation, the phenotyping logic is a convolution neural network (CNN) with a plurality of convolution layers. In another implementation, the phenotyping logic is a recurrent neural network (RNN) such as a long short-term memory network (LSTM), bi-directional LSTM (Bi-LSTM), or a gated recurrent unit (GRU). In yet another implementation, the phenotyping logic includes both a CNN and an RNN.

In yet other implementations, the phenotyping logic can use 1D convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-ch phenotyping logic el convolutions, shuffled grouped convolutions, spatial separable convolutions, and deconvolutions. The phenotyping logic can use one or more loss functions such as logistic regression/log loss, multi-class cross-entropy/softmax loss, binary cross-entropy loss, mean-squared error loss, L1 loss, L2 loss, smooth L1 loss, and Huber loss. The phenotyping logic can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). The phenotyping logic can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential linear unit (ELU), sigmoid and hyperbolic tangent (tanh)), batch normalization layers, regularization layers, dropout, pooling layers (e.g., max or average pooling), global average pooling layers, and attention mechanisms (e.g., self-attention).

The phenotyping logic can be a rule-based model, linear regression model, a logistic regression model, an Elastic Net model, a support vector machine (SVM), a random forest (RF), a decision tree, and a boosted decision tree (e.g., XGBoost), or some other tree-based logic (e.g., metric trees, kd-trees, R-trees, universal B-trees, X-trees, ball trees, locality sensitive hashes, and inverted indexes). The phenotyping logic can be an ensemble of multiple models, in some implementations.

The phenotyping logic is trained using backpropagation-based gradient update techniques. Example gradient descent techniques that can be used for training the phenotyping logic include stochastic gradient descent, batch gradient descent, and mini-batch gradient descent. Some examples of gradient descent optimization algorithms that can be used to train the phenotyping logic are Momentum, Nesterov accelerated gradient, Adagrad, Adadelta, RMSprop, Adam, AdaMax, Nadam, and AMSGrad.

In different implementations, the phenotyping logic includes self-attention mechanisms like Transformer, Vision Transformer (ViT), Bidirectional Transformer (BERT), Detection Transformer (DETR), Deformable DETR, UP-DETR, DeiT, Swin, GPT, iGPT, GPT-2, GPT-3, BERT, SpanBERT, RoBERTa, XLNet, ELECTRA, UniLM, BART, T5, ERNIE (THU), KnowBERT, DeiT-Ti, DeiT-S, DeiT-B, T2T-ViT-14, T2T-ViT-19, T2T-ViT-24, PVT-Small, PVT-Medium, PVT-Large, TNT-S, TNT-B, CPVT-S, CPVT-S-GAP, CPVT-B, Swin-T, Swin-S, Swin-B, Twins-SVT-S, Twins-SVT-B, Twins-SVT-L, Shuffle-T, Shuffle-S, Shuffle-B, XCiT-S12/16, CMT-S, CMT-B, VOLO-D1, VOLO-D2, VOLO-D3, VOLO-D4, MoCo v3, ACT, TSP, Max-DeepLab, VisTR, SETR, Hand-Transformer, HOT-Net, METRO, Image Transformer, Taming transformer, TransGAN, IPT, TTSR, STTN, Masked Transformer, CLIP, DALL-E, Cogview, UniT, ASH, TinyBert, FullyQT, ConvBert, FCOS, Faster R-CNN+FPN, DETR-DCS, TSP-FCOS, TSP-RCNN, ACT+MKDD (L=32), ACT+MKDD (L=16), SMCA, Efficient DETR, UP-DETR, UP-DETR, ViTB/16-FRCNN, ViT-B/16-FRCNN, PVT-Small+RetinaNet, Swin-T+RetinaNet, Swin-T+ATSS, PVT-Small+DETR, TNT-S+DETR, YOLOS-Ti, YOLOS-S, and YOLOS-B.

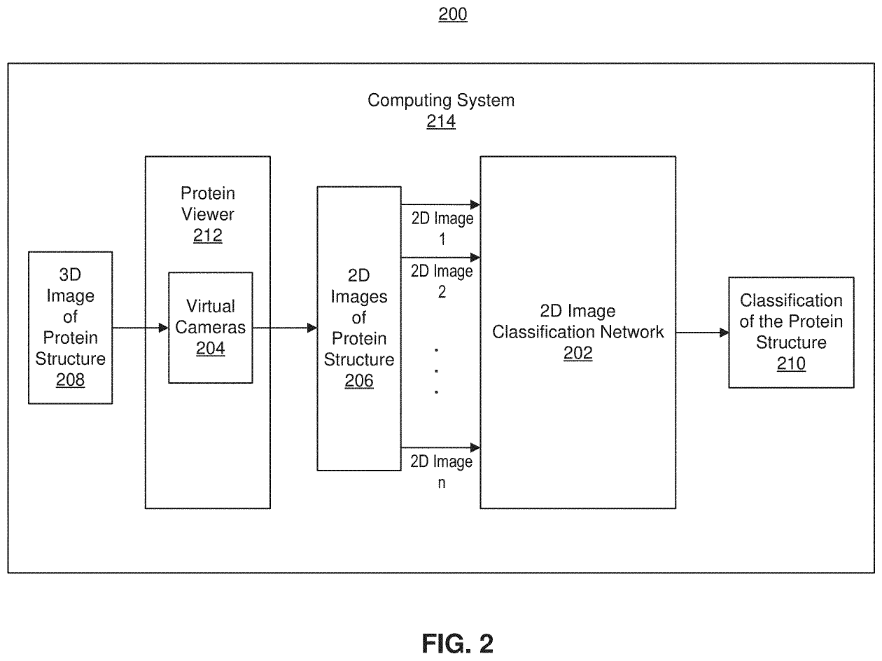

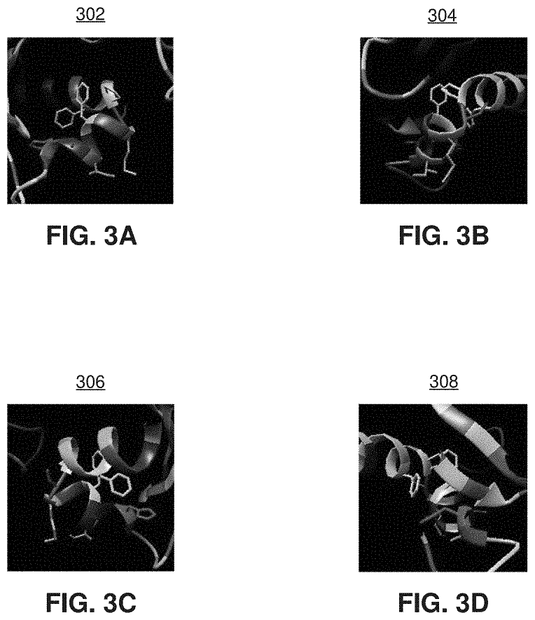

FIG. 2 depicts a system 200, including a 2D image classification network 202 (e.g., a network implemented by convolutional neural networks) that generates classifications of protein structures (e.g., the pathogenicity of the protein structures). The system 200 includes a plurality of virtual cameras 204, configured to generate a plurality of 2D images of a protein structure 206 from a 3D image of the protein structure 208. For example, see the 2D images shown in FIGS. 3A, 3B, 3C, and 3D. Each camera of the plurality of virtual cameras 204 is configured to capture a respective image of the plurality of 2D images of the protein structure 206. The 2D image classification network 202 is configured to process the plurality of 2D images of the protein structure 206 to generate a classification of the protein structure 210.

For the purposes of this disclosure, it is to be understood that a virtual camera is a function of animation software that operates in a way analogous to a camera. The software that includes the virtual camera includes calculations that determines the rending of an object based on the location and angle of the virtual camera according to the software. Analogous to a camera, a virtual camera can use functions such as pan, zoom, focus and changing of focal points.

Also, for the purposes of this disclosure, a protein viewer can include any type of software, with or without virtual cameras, to view proteins or protein structures or view derivatives of proteins or protein structures. Also, it is to be understood for the purpose of this disclosure that a protein structure is a structural part of one or more proteins. For instance, a protein structure can include a part of an amino acid, one or more amino acids, or one or more residues, and a residue is an amino acid once it is linked in a protein chain.

In some implementations, such as the implementation shown in FIG. 2, the plurality of virtual cameras 204 are a part of a protein viewer 212. The protein viewer 212 is configured to select the 3D image of the protein structure 208 and expand (e.g., zoom-in, increase resolution) the 3D image of the protein structure. The protein viewer 212 is also configured to color the 3D image of the protein structure 208 by a coloring parameter of the protein viewer. Also, the protein viewer 212 is configured to capture, via the plurality of virtual cameras 204, the plurality of 2D images of the protein structure 206 from different points of view (such as after the 3D image of the protein structure 208 has been expanded (e.g., zoomed-in) and colored).

In some implementations, such as the implementation shown in FIG. 2, the system 200 includes a computing system 214, configured to train the 2D image classification network 202. To train the 2D image classification network 202, the computing system 214 is configured to repeat, for 3D images of protein structures (e.g., see the 3D image of the protein structure 208), the following steps: (1) generating a plurality of 2D images of a protein structure from a 3D image of the protein structure, and (2) processing, by the 2D image classification network, the plurality of 2D images to generate a classification of the protein structure. To train the 2D image classification network 202, the computing system 214 is also configured to adjust, after each repetition of the steps (1) and (2), parameters of the 2D image classification network 202 according to the generated classification of the protein structure.

In some implementations, the protein structure includes a target amino acid.

In some implementations, the classification of the protein structure includes a pathogenicity score.

In some implementations, the 2D image classification network 202 is configured to process the plurality of 2D images of the protein structure 206 to determine pathogenicity of a nucleotide variant that causes a target amino acid in the protein structure to be replaced with an alternate amino acid. In such implementations, the classification includes the pathogenicity of the nucleotide variant.

In some implementations, the pathogenicity of the nucleotide variant is represented by a pathogenicity score. It is to be understood for the purposes of this disclosure that pathogenicity is the property of causing disease. Thus, a pathogenicity score provides an extent in which something causes disease. In some implementations, the pathogenicity score pertains to an extent in which a nucleotide variant causes disease relative to other nucleotide variants in genomes of a population (such as the human population).

In some implementations, the plurality of virtual cameras 204 is configured to generate a plurality of graphical representations of amino acids in the protein structure from a 3D graphical representation of the amino acids. In such implementations, wherein the 3D image of the protein structure 208 includes the 3D graphical representation of the amino acids. And, each image of the plurality of 2D images of the protein structure 206 includes a respective graphical representation of the amino acids of the plurality of graphical representations of the amino acids.

In some implementations, the amino acids include a target amino acid. In some implementations, the 2D image classification network 202 is configured to process the plurality of 2D images of the protein structure 206 to determine pathogenicity of a nucleotide variant that causes the target amino acid to be replaced with an alternate amino acid. In some of such implementations, the classification of the protein structure includes a pathogenicity score.

Also, as mentioned herein, in some implementations, the plurality of virtual cameras 204 are a part of the protein viewer 212. And, in some implementations wherein the plurality of virtual cameras 204 is configured to generate a plurality of graphical representations of amino acids in the protein structure, the protein viewer 212 is configured to: (1) select the 3D image of the protein structure 208, (2) expand the 3D image of the protein structure 208, (3) color each amino acid of the plurality of graphical representations of amino acids, in the 3D image of the protein structure 208, by amino acid type, and (4) capture, via the plurality of virtual cameras 204, the plurality of 2D images of the protein structure 206 from different points of view (such as after the 3D image has been expanded/zoomed-in and colored).

In some of such implementations wherein the plurality of virtual cameras 204 is configured to generate a plurality of graphical representations of amino acids in the protein structure, the coloring parameter is amino acid type and the protein viewer 212 is configured to color the 3D image of the protein structure 208 by amino acid type, in some other implementations. In other implementations, the coloring parameter is used to tune protein structure elements like atomic distribution.

Also, in some implementations, the coloring parameter is used to represent conservation of protein structure and the protein viewer 212 is configured to color the 3D image of the protein structure 208 by conservation of protein structure. Also, in some of such implementations, the coloring parameter is structural quality and the protein viewer 212 is configured to color the 3D image of the protein structure 208 by structural quality.

Some examples of the structure quality/confidence information include GMQE score (provided by SwissModel); B-factor; temperature factor column of homology models (indicates how well a residue satisfies (physical) constraints in the protein structure); normalized number of aligning template proteins for the residue nearest to the center of a voxel (alignments provided by HHpred, e.g., voxel is nearest to a residue at which 3 of 6 template structures align, signifying that the feature has value 3/6=0.5; minimum, maximum, and mean TM-scores; and predicted TM-scores of the template protein structures that align to the residue that is nearest to a voxel (continuing the example above, assume the 3 template structure has TM-scores 0.5, 0.5 and 1.5, then the minimum is 0.5, the mean is ⅔, and the maximum is 1.5). The TM-scores can be provided per protein template by HHpred.

In some implementations, the 3D image of the protein structure 208 includes graphical representations of atoms. In some implementations, the 3D image of the protein structure 208 includes graphical representations of residues. Also, in some implementations, the 3D image of the protein structure 208 includes graphical representations of atoms and residues.

Appropriate aspects of the system 200 implement any one of the methods described herein. For example, the system 200 can provide a computer-implemented method of determining pathogenicity of variants that includes accessing a structural rendition of amino acids, capturing images of those parts of the structural rendition that contain a target amino acid from the amino acids, and, based on the images, determining pathogenicity of a nucleotide variant that mutates the target amino acid into an alternate amino acid.

FIGS. 3A, 3B, 3C and 3D depict 2D images 302, 304, 306, and 308 of a protein structure generated from a 3D image of a protein structure. As shown with the 2D images 302, 304, 306, and 308, in implementations with a protein viewer (e.g., see protein viewer 212), the viewer is configured to color a 3D image of a protein structure by a coloring parameter of the protein viewer and then capture, such as via a plurality of virtual cameras (e.g., see the plurality of virtual cameras 204), the plurality of 2D images from different points of view. The protein structure colored by the viewer, as shown as the 2D images 302, 304, 306, and 308, includes a target amino acid. And, processing of the plurality of 2D images by some of the implementations disclosed herein include processing, by a 2D image classification network, the plurality of 2D images to determine pathogenicity of a nucleotide variant that causes the target amino acid in the protein structure to be replaced with an alternate amino acid.

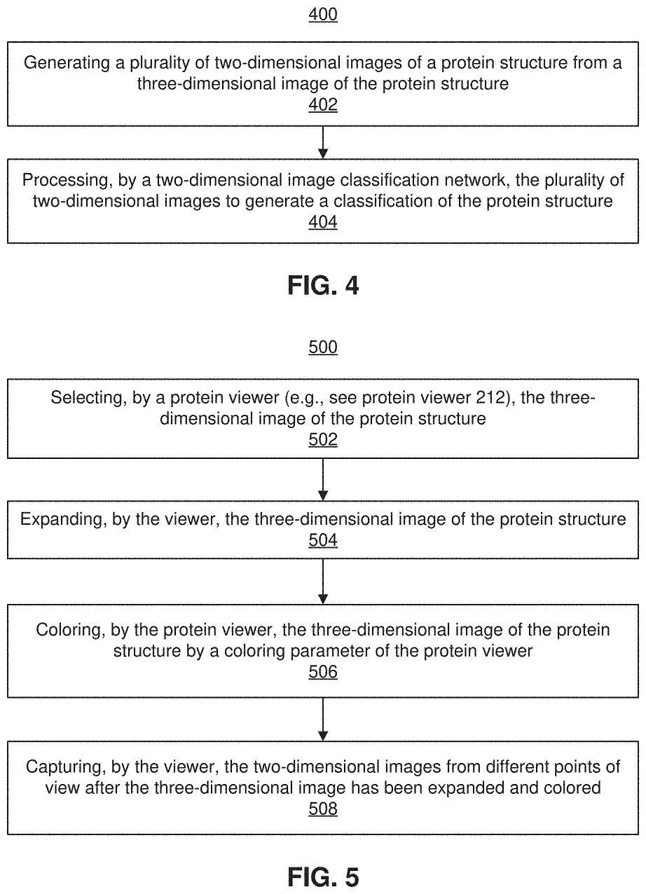

FIGS. 4, 5, and 6 depict methods 400, 500, and 600, respectively. Each of methods 400, 500, and 600 use a 2D image classification network (such as the 2D image classification network 202 shown in FIG. 2) to generate classifications of protein structures. Any one of the 2D image classification networks described herein can be used by the methods 400, 500, and 600.

As shown by FIG. 4, the method 400, at step 402, commences with generating a plurality of 2D images of a protein structure from a 3D image of the protein structure (e.g., see the plurality of 2D images of the protein structure 206 and the 3D image of the protein structure 208 shown in FIG. 2 as well as the 2D images 302, 304, 306, and 308 shown in FIGS. 3A, 3B, 3C, and 3D, respectively). Then, at step 404, the method 400 continues with processing, by a 2D image classification network, the plurality of 2D images to generate a classification of the protein structure. In some implementations of the method 400, the protein structure includes a target amino acid. In some implementations of the method 400, the classification of the protein structure includes generating a pathogenicity score. More specifically, in some implementations of the method 400, the processing of the plurality of 2D images, at step 404, includes processing, by the 2D image classification network, the plurality of 2D images to determine pathogenicity of a nucleotide variant that causes a target amino acid in the protein structure to be replaced with an alternate amino acid. And, in such implementations, the classification of the protein structure can include a pathogenicity score.

In some implementations of the method 400, the generating of the plurality of 2D images includes generating a plurality of graphical representations of amino acids in the protein structure from a 3D graphical representation of the amino acids. The 3D image of the protein structure includes the 3D graphical representation of the amino acids, and each image of the plurality of 2D images includes a respective graphical representation of the amino acids of the plurality of graphical representations of the amino acids. In such implementations, the amino acids include a target amino acid. Also, in some examples, the processing of the plurality of 2D images includes processing, by the 2D image classification network, the plurality of 2D images to determine pathogenicity of a nucleotide variant that causes the target amino acid to be replaced with an alternate amino acid. In such examples, the classification of the protein structure can include a pathogenicity score.

Also, as shown by the steps of method 500 depicted in FIG. 5, in such implementations where the plurality of graphical representations of amino acids of the protein structure is generated as well as other implementations, the generating of the plurality of 2D images can include: (1) selecting, by a protein viewer (e.g., see protein viewer 212), the 3D image of the protein structure (e.g., see step 502), (2) expanding, by the protein viewer, the 3D image of the protein structure (e.g., see step 504), and (3) coloring, by the protein viewer, the 3D image of the protein structure by a coloring parameter of the protein viewer (e.g., see step 506). In some examples, the coloring of the 3D image of the protein structure is by amino acid type. In such instances, the coloring includes coloring each amino acid of the plurality of graphical representations of amino acids by amino acid type. Also, in such implementations where the plurality of graphical representations of amino acids of the protein structure is generated as well as other implementations, the generating of the plurality of 2D images can include (4) capturing, by the protein viewer, the plurality of 2D images from different points of view after the 3D image has been expanded and colored (e.g., see step 508). As mentioned, the coloring parameter can be amino acid type, and in such examples, the coloring of the 3D image of the protein structure is by amino acid type. Also, the coloring parameter can be conservation of protein structure, and in such examples, the coloring of the 3D image of the protein structure is by conservation of protein structure. Also, the coloring parameter can be structural quality, and in such examples, the coloring of the 3D image of the protein structure is by structural quality.

Referring to FIGS. 4 and 5, in some implementations, the step 402 of method 400 can include steps 502 to 508 of method 500. Also, referring to FIGS. 4 and 5, in some implementations of the method 400, the 3D image of the protein structure includes graphical representations of atoms. In some implementations of the methods 400 and 500, the 3D image of the protein structure includes graphical representations of residues. Also, the 3D image of the protein structure can include graphical representations of atoms and residues.

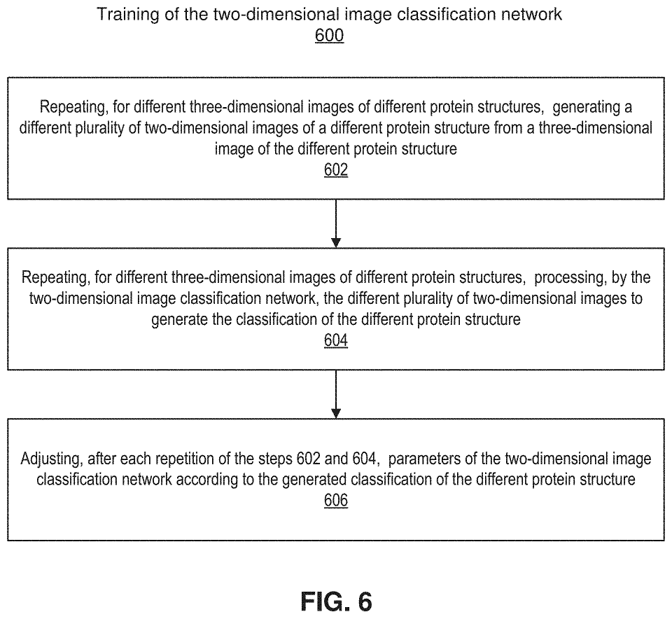

FIG. 6 depicts method 600, which includes training the 2D image classification network. The training of the 2D image classification network includes repeating, for different 3D images of different protein structures, generating a different plurality of 2D images of a different protein structure from a 3D image of the different protein structure (at step 602). The training of the 2D image classification network also includes repeating, for different 3D images of different protein structures, processing, by the 2D image classification network, the different plurality of 2D images to generate the classification of the different protein structure (at step 604). In other words, method 600 includes, repeating the following steps for different 3D images of different protein structures: (1) generating a different plurality of 2D images of a different protein structure from a 3D image of the different protein structure (at step 602), and (2) processing, by the 2D image classification network, the different plurality of 2D images to generate the classification of the different protein structure (at step 604). The training of the 2D image classification network also includes adjusting, after each repetition of the steps 602 and 604, parameters of the 2D image classification network according to the generated classification of the different protein structure (at step 606). In the training shown in method 600, the processing of the plurality of 2D images can include processing, by the 2D image classification network, the plurality of 2D images to determine pathogenicity of a nucleotide variant that causes a target amino acid in the protein structure to be replaced with an alternate amino acid. Also, the classification of the protein structure can include a pathogenicity score.

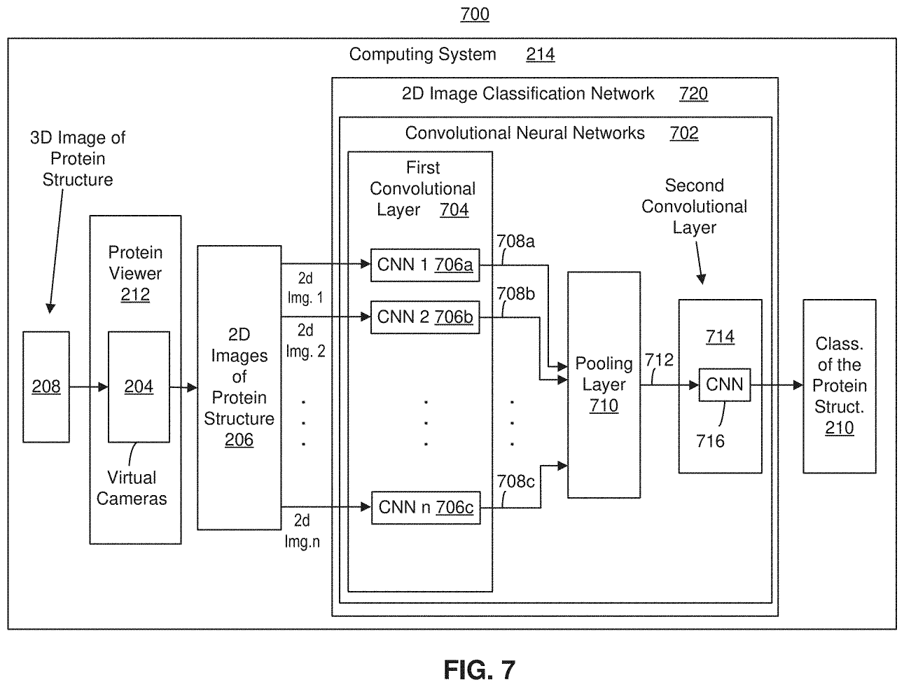

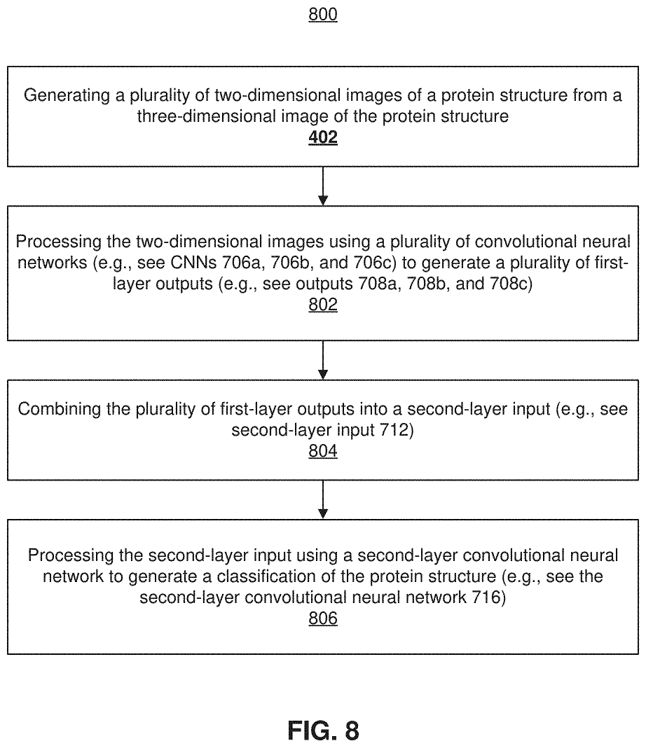

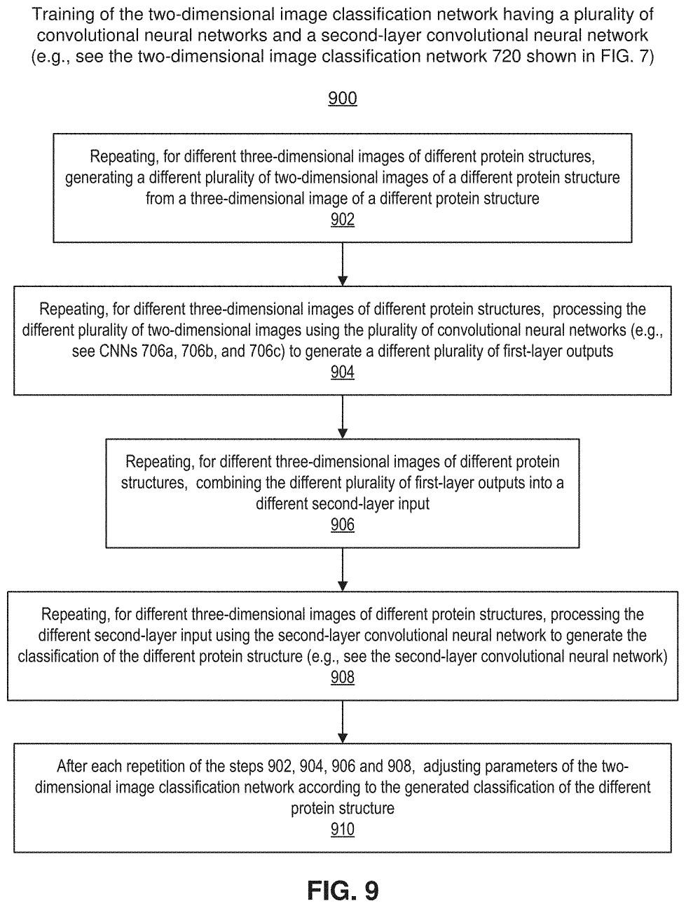

FIG. 7 depicts a system 700 including convolutional neural networks 702. The combination of the convolutional neural networks 702 is configured to generate classifications of protein structures. With respect to some implementations, the convolutional neural networks described herein are multi-view convolutional neural networks for classifying protein structures. In some implementations, the multi-view CNNs are somewhat similar to the multi-view CNNs described in “Multi-view Convolutional Neural Networks for 3D Shape Recognition” by Hang Su et al., published May 5, 2015 for the 2015 IEEE International Conference on Computer Vision (ICCV) (Su, Hang et al. “Multi-view Convolutional Neural Networks for 3D Shape Recognition.” 2015 IEEE International Conference on Computer Vision (ICCV) (2015): 945-953.). In general, for the purposes of this disclosure, a convolutional neural network, or CNN, is a class of artificial neural network that employs an operation called convolution. Convolutional networks are a specialized type of neural networks that use convolution in place of general matrix multiplication in at least one of their layers. In some implementations, CNNs are regularized versions of multilayer perceptrons. Multilayer perceptrons are fully connected networks in that each neuron in one layer is connected to all neurons in the next layer.

Also, the system 700 includes the plurality of virtual cameras 204 (e.g., see FIG. 2), configured to generate the plurality of 2D images of a protein structure 206 from the 3D image of the protein structure 208 (e.g., see FIG. 2 and images 3A, 3B, 3C, and 3D). Each camera of the plurality of virtual cameras 204 is configured to capture a respective image of the plurality of 2D images of the protein structure 206.

As shown in FIG. 7, the convolutional neural networks 702 includes a first convolutional layer 704 including a plurality of convolutional neural networks (e.g., see CNNs 706a, 706b, and 706c), configured to generate a plurality of first-layer outputs (e.g., see outputs 708a, 708b, and 708c) based on the plurality of 2D images of the protein structure 206. Each output of the plurality of first-layer outputs is for a respective image of the plurality of 2D images of the protein structure 206. Each convolutional neural network of the plurality of convolutional neural networks is for a respective image of the plurality of 2D images.

The convolutional neural networks 702 also includes a pooling layer 710, configured to combine the plurality of first-layer outputs into a second-layer input 712. Also, the convolutional neural networks 702 includes a second convolutional layer 714 including a second-layer convolutional neural network 716, configured to generate the classification of the protein structure 210 based on the second-layer input 712.

As shown in FIG. 7, in some implementations of the system 700, the plurality of virtual cameras 204 are a part of the protein viewer 212. Similar to the system 200, with some implementations of system 700, the protein viewer 212 can be configured to: (1) select the 3D image of the protein structure 208, (2) expand the 3D image of the protein structure, (3) color each amino acid of the plurality of graphical representations of amino acids, in the 3D image of the protein structure, by amino acid type, and (4) capture, via the plurality of virtual cameras 204, the plurality of 2D images of the protein structure 206 from different points of view (such as after the 3D image has been expanded and colored). In a more generalized implementation, the plurality of virtual cameras are a part of the protein viewer, and the protein viewer is configured to: (1) select the 3D image of the protein structure, (2) expand the 3D image of the protein structure, (3) color the 3D image of the protein structure by a coloring parameter of the protein viewer, and (4) capture, via the plurality of virtual cameras, the plurality of 2D images from different points of view (such as after the 3D image has been expanded and colored).

As mentioned, in some implementations of the system 700, the coloring parameter is amino acid type, and the coloring of the 3D image of the protein structure is by amino acid type. Also, the coloring parameter can be conservation of protein structure, and the coloring of the 3D image of the protein structure can be by conservation of protein structure. Further, in some examples, the coloring parameter is structural quality, and the coloring of the 3D image of the protein structure is by structural quality.

Also, as shown in FIG. 7, in some implementations of the system 700, the system includes a 2D image classification network 720, including the first convolutional layer 704, the pooling layer 710, and the second convolutional layer 714. The system 700 also includes the computing system 214, configured to train the 2D image classification network 720. In such implementations and others, the computing system 214 is configured to adjust parameters of the 2D image classification network 720. For example, the computing system 214 can be configured to adjust parameters of the plurality of convolutional neural networks (e.g., see CNNs 706a, 706b, and 706c) in the first convolutional layer 704. Also, for example, the computing system 214 can be configured to adjust parameters of the second-layer convolutional neural network 716 in the second convolutional layer 714. Also, for example, the computing system 214 can be configured to adjust parameters of the pooling layer 710. Such adjustments can occur in the training of the 2D image classification network 720, performed by the computing system 214, or in some other process performed by the computing system.

Specifically, in some implementations, the computing system 214 is configured to train the 2D image classification network 720 by being configured to repeat, for different 3D images of different protein structures, the following steps: (1) generating, via the plurality of virtual cameras 204, a different plurality of 2D images of a different protein structure from a 3D image of a different protein structure, (2) generating, via the plurality of convolutional neural networks (e.g., see CNNs 706a, 706b, and 706c), a different plurality of first-layer outputs based on the different plurality of 2D images, (3) combining, via the pooling layer 710, the different plurality of first-layer outputs into a different second-layer input, and (4) generating, via the second-layer convolutional neural network 716, a classification of the different protein structure based on the different second-layer input. The computing system 214 is also configured to train the 2D image classification network 720 by being configured to adjust, after each repetition of the steps (1), (2), (3) and (4), parameters of the 2D image classification network 720 according to the generated classification of the different protein structure.

Similar to system 200, with the system 700, the protein structure can include a target amino acid, and the classification of the protein structure can include a pathogenicity score. In such implementations. Also, for example, the 2D image classification network 202 can be configured to process the plurality of 2D images to determine pathogenicity of a nucleotide variant that causes the target amino acid to be replaced with an alternate amino acid.

Also, similar to system 200, with the system 700, the plurality of virtual cameras 204 can be configured to generate a plurality of graphical representations of amino acids in the protein structure from a 3D graphical representation of the amino acids. Also, the 3D image of the protein structure includes the 3D graphical representation of the amino acids, and each image of the plurality of 2D images includes a respective graphical representation of the amino acids of the plurality of graphical representations of the amino acids. Further, in such implementations, the amino acids include a target amino acid. Additionally, in such implementations, the system 700 includes the 2D image classification network 720 (instead of the network 202) and the 2D image classification network 720 can be configured to process the plurality of 2D images to determine pathogenicity of a nucleotide variant that causes the target amino acid to be replaced with an alternate amino acid. Also, in such implementations, the classification of the protein structure can include a pathogenicity score.

Also, similar to system 200, with the system 700, the 3D image of the protein structure can include graphical representations of atoms. And, the 3D image of the protein structure can include graphical representations of residues.

Appropriate aspects of the system 700 implement, such a through a computer-implemented method, any one of the methods described herein using CNNs. For example, the system 700 can provide a computer-implemented method of determining pathogenicity of variant that includes accessing a structural rendition of amino acids, capturing images of those parts of the structural rendition that contain a target amino acid from the amino acids, and, based on the images, determining pathogenicity, via CNNs, of a nucleotide variant that mutates the target amino acid into an alternate amino acid.

FIGS. 8, 9, and 10 depict methods 800, 900, and 1000, respectively. Each of the methods 800, 900, and 1000 use convolutional neural networks to generate classifications of protein structures (such as the convolutional neural networks 702 shown in FIG. 7, which are a part of the 2D image classification network 720) to generate classifications of protein structures. Any one of the 2D image classification networks described herein that includes convolutional neural networks can be used by the methods 800, 900, and 1000.

As shown by FIG. 8, the method 800 commences with generating a plurality of 2D images of a protein structure from a 3D image of the protein structure at step 402 (which is also in method 400). E.g., see the plurality of 2D images of the protein structure 206 and the 3D image of the protein structure 208 shown in FIG. 2 as well as the 2D images 302, 304, 306, and 308 shown in FIGS. 3A, 3B, 3C, and 3D, respectively. Next, at step 802, the method 800 continues with processing the plurality of 2D images using a plurality of convolutional neural networks (e.g., see CNNs 706a, 706b, and 706c) to generate a plurality of first-layer outputs (e.g., see outputs 708a, 708b, and 708c). Each output of the plurality of first-layer outputs is for a respective image of the plurality of 2D images, and each convolutional neural network of the plurality of convolutional neural networks is for a respective image of the plurality of 2D images.

Subsequent to the generation the plurality of first-layer outputs at step 802, the method 800 continues with combining the plurality of first-layer outputs into a second-layer input at step 804 (e.g., see second-layer input 712). And, at step 806, the method 800 continues with processing the second-layer input using a second-layer convolutional neural network to generate a classification of the protein structure. E.g., see the second-layer convolutional neural network 716.

In some implementations of the method 800, the protein structure includes a target amino acid. In some implementations of the method 800, the classification of the protein structure includes a pathogenicity score.

In some implementations of the method 800, the processing of the plurality of 2D images includes processing, by a 2D image classification network, the plurality of 2D images to determine pathogenicity of a nucleotide variant that causes a target amino acid in the protein structure to be replaced with an alternate amino acid. And, in such implementations, the 2D image classification network includes the plurality of convolutional neural networks and the second-layer convolutional neural network. And, in such implementations, the classification of the protein structure includes a pathogenicity score.

In some implementations of the method 800, similar to some implementations of the method 400, the generating of the plurality of 2D images at step 402 includes generating a plurality of graphical representations of amino acids in the protein structure from a 3D graphical representation of the amino acids. In such implementations, the 3D image of the protein structure includes the 3D graphical representation of the amino acids, and each image of the plurality of 2D images includes a respective graphical representation of the amino acids of the plurality of graphical representations of the amino acids. Also, in such implementations, the amino acids can include a target amino acid. And, in such implementations, the processing of the plurality of 2D images can include processing, by a 2D image classification network, the plurality of 2D images to determine pathogenicity of a nucleotide variant that causes the target amino acid to be replaced with an alternate amino acid. In such examples and others, the 2D image classification network includes the plurality of convolutional neural networks and the second-layer convolutional neural network. And, the classification of the protein structure includes a pathogenicity score.

FIG. 9 depicts method 900, which includes training a 2D image classification network having a plurality of convolutional neural networks and a second-layer convolutional neural network (e.g., see the 2D image classification network 720 shown in FIG. 7). The training of such a 2D image classification network includes repeating, for different 3D images of different protein structures, generating a different plurality of 2D images of a different protein structure from a 3D image of a different protein structure (at step 902). The training of the 2D image classification network also includes repeating, for different 3D images of different protein structures, processing the different plurality of 2D images using the plurality of convolutional neural networks (e.g., see CNNs 706a, 706b, and 706c) to generate a different plurality of first-layer outputs (at step 904). The training of the 2D image classification network also includes repeating, for different 3D images of different protein structures, combining the different plurality of first-layer outputs into a different second-layer input (at step 906). The training of the 2D image classification network also includes repeating, for different 3D images of different protein structures, processing the different second-layer input using the second-layer convolutional neural network to generate the classification of the different protein structure (at step 908). E.g., see the second-layer convolutional neural network 716.

In other words, method 900 includes, repeating the following steps for different 3D images of different protein structures: (1) generating a different plurality of 2D images of a different protein structure from a 3D image of a different protein structure (at step 902), (2) processing the different plurality of 2D images using the plurality of convolutional neural networks to generate a different plurality of first-layer outputs (at step 902), (3) combining the different plurality of first-layer outputs into a different second-layer input (at step 906), and (4) processing the different second-layer input using the second-layer convolutional neural network to generate the classification of the different protein structure (at step 908).

In method 900, the training of the 2D image classification network also includes, after each repetition of the steps 902, 904, 906 and 908, adjusting parameters of the 2D image classification network according to the generated classification of the different protein structure (at step 910). Also, in the training shown in method 900, the processing of the plurality of 2D images can include processing, by the 2D image classification network, the plurality of 2D images to determine pathogenicity of a nucleotide variant that causes a target amino acid in the protein structure to be replaced with an alternate amino acid. Also, the classification of the protein structure can include a pathogenicity score.

In some implementations of method 900, the adjusting of the parameters of the 2D image classification network at step 910 includes adjusting parameters of the plurality of convolutional neural networks. In some implementations of method 900, the adjusting of the parameters of the 2D image classification network at step 910 includes adjusting parameters of the second-layer convolutional neural network. In some implementations of method 900, the adjusting of the parameters of the 2D image classification network at step 910 includes adjusting parameters of a pooling layer. And, in some of such implementations, the pooling layer performs the combining of the different plurality of first-layer outputs into the second-layer input at step 906.

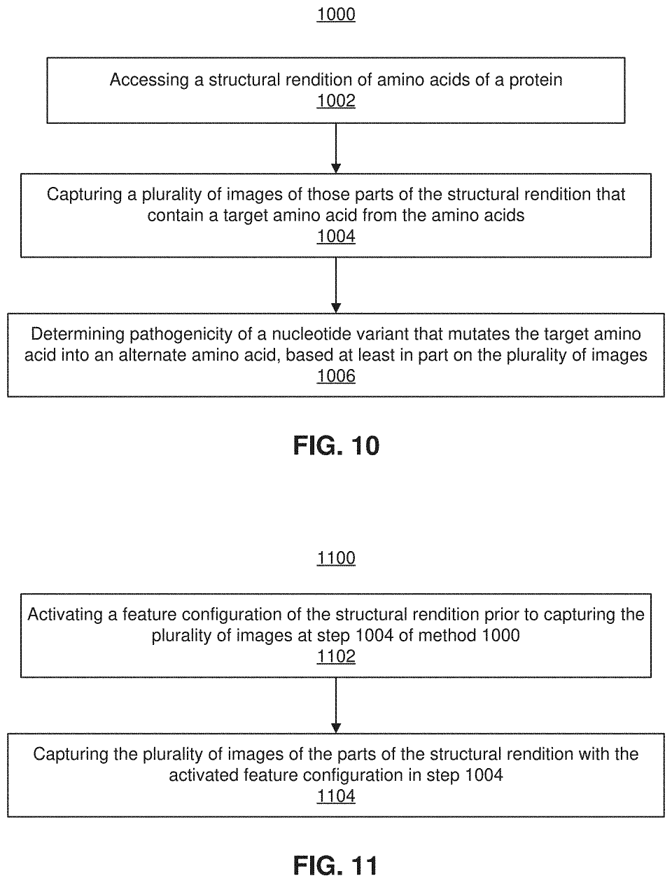

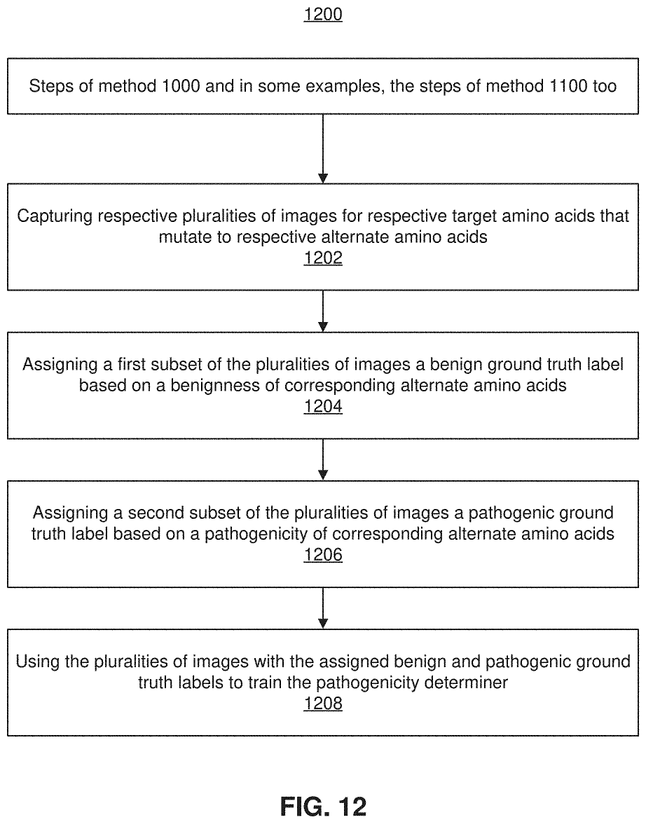

FIGS. 10, 11, and 12 depict methods 1000, 1100, and 1200, respectively, which are for determining pathogenicity of variants, such as nucleotide variants.

It is to be understood for the purposes of this disclosure that variants are not necessarily limited to nucleotide variants. And, a nucleotide variant can include a variant of one or more nucleotides. And, unless specified otherwise herein, the term “variant” refers to a nucleic acid sequence that is different from a nucleic acid reference. Typical nucleic acid sequence variant includes without limitation single nucleotide polymorphism (SNP), short deletion and insertion polymorphisms (indel), copy number variation (CNV), microsatellite markers or short tandem repeats and structural variation. In this application, amino acid substitutions include single and/or multiple amino acid substitutions.

The method 1000 commences with accessing a structural rendition of amino acids of a protein (at step 1002). The method 1000 continues with capturing a plurality of images of those parts of the structural rendition that contain a target amino acid from the amino acids (at step 1004). Finally, the method 1000 ends with determining pathogenicity of a nucleotide variant that mutates the target amino acid into an alternate amino acid, based at least in part on the plurality of images (at step 1006).

In some implementations of method 1000, images in the plurality of images are captured from multiple perspectives. For example, the images are captured from multiple points of view, multiple orientations, multiple positions, multiple zoom levels, or the like, or some combination thereof.

In some implementations of the method 1000, the parts of the structural rendition contain the target amino acid and some additional amino acids adjacent to the target amino acid. Also, in some implementations of the method 1000, the structural rendition is a 3D structural rendition.

The method 1100 includes steps 1102 and 1104, which can be combined with some steps of method 1000. The step 1102 includes activating a feature configuration of the structural rendition prior to capturing the plurality of images, at step 1004 of method 1000. And, capturing the plurality of images of the parts of the structural rendition with the activated feature configuration in step 1004, at step 1104.

In some implementations of method 1100, the feature configuration is displaying a residue detail of the amino acids. In some implementations of method 1100, the feature configuration is displaying an atomic detail of the amino acids. In some implementations of method 1100, the feature configuration is displaying a polymer detail of the amino acids. In some implementations of method 1100, the feature configuration is displaying a ligand detail of the amino acids. In some implementations of method 1100, the feature configuration is displaying a chain detail of the amino acids. In some implementations of method 1100, the feature configuration is displaying surface effects of the amino acids. In such examples, the surface effects include transparency and color coding for electrostatic and hydrophobic values. In some implementations of method 1100, the feature configuration is displaying density maps of the amino acids. In some implementations of method 1100, the feature configuration is displaying supramolecular assemblies of the amino acids. In some implementations of method 1100, wherein the feature configuration is displaying sequence alignments of the amino acids. In some implementations of method 1100, wherein the feature configuration is displaying docking results of the amino acids. In some implementations of method 1100, the feature configuration is displaying trajectories of the amino acids. In some implementations of method 1100, the feature configuration is displaying conformational ensembles of the amino acids. In some implementations of method 1100, the feature configuration is displaying secondary structures of the amino acids. In some implementations of method 1100, the feature configuration is displaying tertiary structures of the amino acids. In some implementations of method 1100, the feature configuration is displaying quaternary structures of the amino acids. In some implementations of method 1100, the feature configuration is setting a zoom factor for displaying the structural rendition. In some implementations of method 1100, the feature configuration is setting a lighting condition for displaying the structural rendition. In some implementations of method 1100, the feature configuration is setting a visibility range for displaying the structural rendition. In some implementations of method 1100, the feature configuration is color coding the structural rendition by the amino acids. In some implementations of method 1100, the feature configuration is color coding the structural rendition by evolutionary conservations of the amino acids. In some implementations of method 1100, the feature configuration is color coding the structural rendition by structural qualities of the amino acids. In some implementations of method 1100, the feature configuration is color coding the structural rendition to identify a gap/missing amino acid.

In some implementations of method 1100, a pathogenicity determiner determines the pathogenicity of the nucleotide variant by processing, as input, the plurality of images, and generating, as output, a pathogenicity score for the alternate amino acid. In some of such examples, the pathogenicity determiner is a neural network. In some of such examples, the neural network is a convolutional neural network. In some of such examples using a CNN, the pathogenicity determiner processes respective images in the plurality of images through respective CNNs to generate respective feature maps for the respective images. Also, in some examples, the respective convolutional neural networks are respective instances of a same convolutional neural network. Also, in some examples, the pathogenicity determiner combines the respective feature maps into a pooled feature map and processes the pooled feature map through a final convolutional neural network to generate the pathogenicity score for the alternate amino acid.

Also, in some examples, the respective images in the plurality of images are combined into a combined representation for processing by the pathogenicity determiner. And, in some of such example, the respective images are arranged as respective color channels in the combined representation. Also, intensity values in the respective images can be pixel-wise summed into pixels of the combined representation.

The method 1200 includes the steps of method 1000 (and in some implementations, the steps of method 1100 too) as well as steps 1202, 1204, 1206, and 1208. One of the purposes of method 1200 is to train the pathogenicity determiner from method 1000. Also, such steps can be used to train any of the 2D image classification networks described herein. The step 1202 includes capturing respective pluralities of images for respective target amino acids that mutate to respective alternate amino acids. The step 1204 includes assigning a first subset of the pluralities of images a benign ground truth label based on a benignness of corresponding alternate amino acids. In other words, the step 1204 includes assigning a first subset of the pluralities of images a benign ground truth label when corresponding alternate amino acids are benign. The step 1206 includes assigning a second subset of the pluralities of images a pathogenic ground truth label based on a pathogenicity of corresponding alternate amino acids. The step 1208 includes using the pluralities of images with the assigned benign and pathogenic ground truth labels to train the pathogenicity determiner.

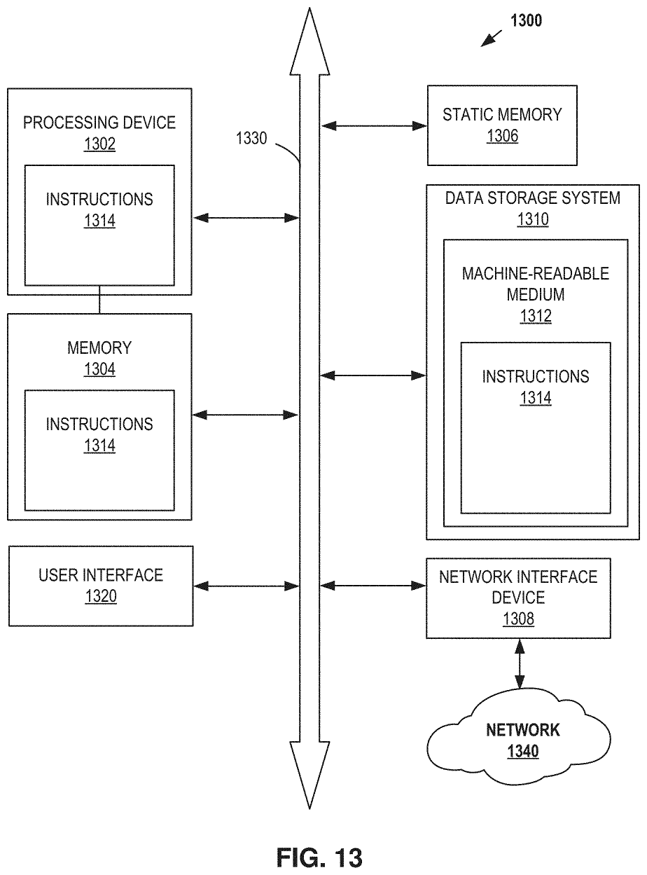

FIG. 13 shows a block diagram of example aspects of the computing system 1300, which can include, be or be a part of any one of the electronic or computing systems described herein. FIG. 13 illustrates parts of the computing system 1300 within which a set of instructions, for causing the machine to perform any one or more of the methodologies discussed herein, are executed.

In some implementations, the computing system 1300 corresponds to a host system that includes, is coupled to, or utilizes memory or is used to perform the operations performed by any one of the computing devices, data processors, and user interface devices described herein. In alternative implementations, the machine is connected (e.g., networked) to other machines in a LAN, an intranet, an extranet, or the Internet. In some implementations, the machine operates in the capacity of a server or a client machine in client-server network environment, as a peer machine in a peer-to-peer (or distributed) network environment, or as a server or a client machine in a cloud computing infrastructure or environment. In some implementations, the machine is a personal computer (PC), a tablet PC, a cellular telephone, a web appliance, a server, or any machine capable of executing a set of instructions (sequential or otherwise) that specify actions to be taken by that machine. Further, while a single machine is illustrated, the term “machine” shall also be taken to include any collection of machines that individually or jointly execute a set (or multiple sets) of instructions to perform any one or more of the methodologies discussed herein.

The computing system 1300 includes a processing device 1302, a main memory 1304 (e.g., read-only memory (ROM), flash memory, dynamic random-access memory (DRAM), etc.), a static memory 1306 (e.g., flash memory, static random-access memory (SRAM), etc.), and a data storage system 1310, which communicate with each other via a bus 1330. The processing device 1302 represents one or more general-purpose processing devices such as a microprocessor, a central processing unit, or the like. More particularly, the processing device is a microprocessor or a processor implementing other instruction sets, or processors implementing a combination of instruction sets. Or, the processing device 1302 is one or more special-purpose processing devices such as an application specific integrated circuit (ASIC), a field programmable gate array (FPGA), a digital signal processor (DSP), network processor, or the like. The processing device 1302 is configured to execute instructions 1314 for performing the operations or steps discussed herein. In some implementations, the computing system 1300 includes a network interface device 1308 to communicate over a communications network 1340 shown in FIG. 13.

The data storage system 1310 includes a machine-readable storage medium 1312 (also known as a computer-readable medium) on which is stored one or more sets of instructions 1314 or software embodying any one or more of the methodologies or functions described herein. The instructions 1314 also reside, completely or at least partially, within the main memory 1304 or within the processing device 1302 during execution thereof by the computing system 1300, the main memory 1304 and the processing device 1302 also constituting machine-readable storage media.

In some implementations, the instructions 1314 include instructions to implement functionality corresponding to any one of the computing devices, data processors, user interface devices, and I/O devices described herein. While the machine-readable storage medium 1312 is shown in an example implementation to be a single medium, the term “machine-readable storage medium” should be taken to include a single medium or multiple media that store the one or more sets of instructions. The term “machine-readable storage medium” shall also be taken to include any medium that is capable of storing or encoding a set of instructions for execution by the machine and that cause the machine to perform any one or more of the methodologies of the present disclosure. The term “machine-readable storage medium” shall accordingly be taken to include solid-state memories, optical media, magnetic media, or the like.

Also, as shown, computing system 1300 includes user interface 1320 that includes a display, in some implementations, and, for example, implements functionality corresponding to any one of the user interface devices disclosed herein. A user interface, such as user interface 1320, or a user interface device described herein includes any space or equipment where interactions between humans and machines occur. A user interface described herein allows operation and control of the machine from a human user, while the machine simultaneously provides feedback information to the user. Examples of a user interface (UI), or user interface device include the interactive aspects of computer operating systems (such as graphical user interfaces or GUI), machinery operator controls, and process controls. A UI described herein includes one or more layers, including a human-machine interface (HMI) that interfaces machines with physical input hardware and output hardware.

Also, it is to be understood, that the methodologies discussed herein are computer-implemented methods and, in some implementations, are implementable by the computing system 1300. For instance, a computer-implemented method includes processing, by an artificial neural network (ANN), a plurality of missense variants to generate a plurality of missense scores pertaining to respective pathogenicity for each missense variant of the plurality of missense variants. Also, the computer-implemented method includes processing, by the ANN, a plurality of indels to generate a plurality of indel scores pertaining to respective pathogenicity for each indel of the plurality of indels and further processing the plurality of indel scores and the plurality of missense scores to be applied to one or more curve-forming functions. And, the computer-implemented method includes applying the further processed scores to the curve-forming function(s) to generate a respective indel curve and a respective missense curve and determining a difference between the respective indel curve and the respective missense curve. Also, the computer-implemented method includes determining one or more scaling functions to reduce the difference between the curves and changing the ANN or an output of the ANN according to the scaling function(s). The changing the ANN or the output of the ANN according to the scaling function(s) includes enhancing the plurality of indel scores according to the scaling function(s) to provide increased accuracy of pathogenicity for each indel of the plurality of indels.

Some portions of the preceding detailed descriptions have been presented in terms of models and symbolic representations of operations on data bits within a computer memory. These modelic descriptions and representations are the ways used by those skilled in the data processing arts to most effectively convey the substance of their work to others skilled in the art. An model is here, and generally, conceived to be a self-consistent sequence of operations leading to a predetermined result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

It should be borne in mind, however, that these and similar terms are to be associated with the appropriate physical quantities and are merely convenient labels applied to these quantities. The present disclosure can refer to the action and processes of a computing system, or similar electronic computing device, which manipulates and transforms data represented as physical (electronic) quantities within the computing system's registers and memories into other data similarly represented as physical quantities within the computing system memories or registers or other such information storage systems.

The present disclosure also relates to an apparatus for performing the operations herein. This apparatus can be specially constructed for the intended purposes, or it can include a general-purpose computer selectively activated or reconfigured by a computer program stored in the computer. Such a computer program can be stored in a computer readable storage medium, such as any type of disk including optical disks, CD-ROMs, and magnetic-optical disks, read-only memories (ROMs), random access memories (RAMs), EPROMs, EEPROMs, magnetic or optical cards, or any type of media suitable for storing electronic instructions, coupled to a computing system bus.

The models and displays presented herein are not inherently related to any particular computer or other apparatus. Various general-purpose systems can be used with programs in accordance with the teachings herein, or it can prove convenient to construct a more specialized apparatus to perform the method. The structure for a variety of these systems will appear as set forth in the description below. In addition, the present disclosure is not described with reference to any particular programming language. It will be appreciated that a variety of programming languages can be used to implement the teachings of the disclosure as described herein.

The present disclosure can be provided as a computer program product, or software, which can include a machine-readable medium having stored thereon instructions, which can be used to program a computing system (or other electronic devices) to perform a process according to the present disclosure. A machine-readable medium includes any mechanism for storing information in a form readable by a machine (e.g., a computer). In some implementations, a machine-readable (e.g., computer-readable) medium includes a machine (e.g., a computer) readable storage medium such as a read only memory (“ROM”), random access memory (“RAM”), magnetic disk storage media, optical storage media, flash memory components, etc.

While the invention has been described in conjunction with the specific implementations described herein, it is evident that many alternatives, combinations, modifications and variations are apparent to those skilled in the art. Accordingly, the example implementations of the invention, as set forth herein are intended to be illustrative only, and not in a limiting sense. Various changes can be made without departing from the spirit and scope of the invention.

“Logic” (e.g., curve-forming functions), as used herein, can be implemented in the form of a computer product including a non-transitory computer readable storage medium with computer usable program code for performing the method steps described herein. The “logic” can be implemented in the form of an apparatus including a memory and at least one processor that is coupled to the memory and operative to perform exemplary method steps. The “logic” can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media). In one implementation, the logic implements a data processing function. The logic can be a general purpose, single core or multicore, processor with a computer program specifying the function, a digital signal processor with a computer program, configurable logic such as an FPGA with a configuration file, a special purpose circuit such as a state machine, or any combination of these. Also, a computer program product can embody the computer program and configuration file portions of the logic.

### CLAUSES

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

One or more implementations and clauses of the technology disclosed or elements thereof can be implemented in the form of a computer product, including a non-transitory computer readable storage medium with computer usable program code for performing the method steps indicated. Furthermore, one or more implementations and clauses of the technology disclosed or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

We disclose the following clauses:

1. A computer-implemented method, comprising:

generating a plurality of two-dimensional images of a protein structure from a three-dimensional image of the protein structure; and

processing, by a two-dimensional image classification network, the plurality of two-dimensional images to generate a classification of the protein structure.

2. The computer-implemented method of clause 1, wherein the protein structure comprises a target amino acid.

3. The computer-implemented method of clause 1, wherein the classification of the protein structure comprises a pathogenicity score.

4. The computer-implemented method of clause 1, wherein the processing of the plurality of two-dimensional images comprises processing, by the two-dimensional image classification network, the plurality of two-dimensional images to determine pathogenicity of a nucleotide variant that causes a target amino acid in the protein structure to be replaced with an alternate amino acid.

5. The computer-implemented method of clause 4, wherein the classification of the protein structure comprises a pathogenicity score.

6. The computer-implemented method of clause 1,

wherein the generating of the plurality of two-dimensional images comprises generating a plurality of graphical representations of amino acids in the protein structure from a three-dimensional graphical representation of the amino acids,

wherein the three-dimensional image of the protein structure comprises the three-dimensional graphical representation of the amino acids, and

wherein each image of the plurality of two-dimensional images comprises a respective graphical representation of the amino acids of the plurality of graphical representations of the amino acids.

7. The computer-implemented method of clause 6, wherein the amino acids comprise a target amino acid.

8. The computer-implemented method of clause 7, wherein the processing of the plurality of two-dimensional images comprises processing, by the two-dimensional image classification network, the plurality of two-dimensional images to determine pathogenicity of a nucleotide variant that causes the target amino acid to be replaced with an alternate amino acid.

9. The computer-implemented method of clause 8, wherein the classification of the protein structure comprises a pathogenicity score.

10. The computer-implemented method of clause 8, wherein the generating of the plurality of two-dimensional images comprises:

selecting, by a protein viewer, the three-dimensional image of the protein structure;

expanding, by the protein viewer, the three-dimensional image of the protein structure;

coloring, by the protein viewer, the three-dimensional image of the protein structure by amino acid type, which comprises coloring each amino acid of the plurality of graphical representations of amino acids by amino acid type; and

capturing, by the protein viewer, the plurality of two-dimensional images from different points of view after the three-dimensional image has been expanded and colored.

11. The computer-implemented method of clause 1, wherein the generating of the plurality of two-dimensional images comprises:

selecting, by a protein viewer, the three-dimensional image of the protein structure;

expanding, by the protein viewer, the three-dimensional image of the protein structure;

coloring, by the protein viewer, the three-dimensional image of the protein structure by a coloring parameter of the protein viewer; and

capturing, by the protein viewer, the plurality of two-dimensional images from different points of view after the three-dimensional image has been expanded and colored.

12. The computer-implemented method of clause 11, wherein the coloring parameter is amino acid type, and wherein the coloring of the three-dimensional image of the protein structure is by amino acid type.

13. The computer-implemented method of clause 11, wherein the coloring parameter is conservation of protein structure, and wherein the coloring of the three-dimensional image of the protein structure is by conservation of protein structure.

14. The computer-implemented method of clause 11, wherein the coloring parameter is structural quality, and wherein the coloring of the three-dimensional image of the protein structure is by structural quality.

15. The computer-implemented method of clause 11, wherein the three-dimensional image of the protein structure comprises graphical representations of atoms.

16. The computer-implemented method of clause 11, wherein the three-dimensional image of the protein structure comprises graphical representations of residues.

17. The computer-implemented method of clause 11, wherein the three-dimensional image of the protein structure comprises graphical representations of atoms and residues.

18. The computer-implemented method of clause 1, further comprising training the two-dimensional image classification network by:

repeating, for different three-dimensional images of different protein structures, the following steps:
(1) generating a different plurality of two-dimensional images of a different protein structure from a three-dimensional image of the different protein structure; and
(2) processing, by the two-dimensional image classification network, the different plurality of two-dimensional images to generate the classification of the different protein structure; and

adjusting, after each repetition of the steps (1) and (2), parameters of the two-dimensional image classification network according to the generated classification of the different protein structure.

19. The computer-implemented method of clause 18, wherein the processing of the plurality of two-dimensional images comprises processing, by the two-dimensional image classification network, the plurality of two-dimensional images to determine pathogenicity of a nucleotide variant that causes a target amino acid in the protein structure to be replaced with an alternate amino acid.

20. The computer-implemented method of clause 19, wherein the classification of the protein structure comprises a pathogenicity score.

21. A computer-implemented method, comprising:

generating a plurality of two-dimensional images of a protein structure from a three-dimensional image of the protein structure;

processing the plurality of two-dimensional images using a plurality of convolutional neural networks to generate a plurality of first-layer outputs,

wherein each output of the plurality of first-layer outputs is for a respective image of the plurality of two-dimensional images, and

wherein each convolutional neural network of the plurality of convolutional neural networks is for a respective image of the plurality of two-dimensional images;

combining the plurality of first-layer outputs into a second-layer input; and

processing the second-layer input using a second-layer convolutional neural network to generate a classification of the protein structure.

22. The computer-implemented method of clause 21, wherein the protein structure comprises a target amino acid.

23. The computer-implemented method of clause 21, wherein the classification of the protein structure comprises a pathogenicity score.

24. The computer-implemented method of clause 21, wherein the processing of the plurality of two-dimensional images comprises processing, by a two-dimensional image classification network, the plurality of two-dimensional images to determine pathogenicity of a nucleotide variant that causes a target amino acid in the protein structure to be replaced with an alternate amino acid, and wherein the two-dimensional image classification network comprises the plurality of convolutional neural networks and the second-layer convolutional neural network.

25. The computer-implemented method of clause 24, wherein the classification of the protein structure comprises a pathogenicity score.

26. The computer-implemented method of clause 21,

wherein the generating of the plurality of two-dimensional images comprises generating a plurality of graphical representations of amino acids in the protein structure from a three-dimensional graphical representation of the amino acids,

wherein the three-dimensional image of the protein structure comprises the three-dimensional graphical representation of the amino acids, and

wherein each image of the plurality of two-dimensional images comprises a respective graphical representation of the amino acids of the plurality of graphical representations of the amino acids.

27. The computer-implemented method of clause 26, wherein the amino acids comprise a target amino acid.

28. The computer-implemented method of clause 27, wherein the processing of the plurality of two-dimensional images comprises processing, by a two-dimensional image classification network, the plurality of two-dimensional images to determine pathogenicity of a nucleotide variant that causes the target amino acid to be replaced with an alternate amino acid, and wherein the two-dimensional image classification network comprises the plurality of convolutional neural networks and the second-layer convolutional neural network.

29. The computer-implemented method of clause 28, wherein the classification of the protein structure comprises a pathogenicity score.

30. The computer-implemented method of clause 28, wherein the generating of the plurality of two-dimensional images comprises:

selecting, by a protein viewer, the three-dimensional image of the protein structure;

expanding, by the protein viewer, the three-dimensional image of the protein structure;

coloring, by the protein viewer, the three-dimensional image of the protein structure by amino acid type, which comprises coloring each amino acid of the plurality of graphical representations of amino acids by amino acid type; and

capturing, by the protein viewer, the plurality of two-dimensional images from different points of view after the three-dimensional image has been expanded and colored.

31. The computer-implemented method of clause 21, wherein the generating of the plurality of two-dimensional images comprises:

selecting, by a protein viewer, the three-dimensional image of the protein structure;

expanding, by the protein viewer, the three-dimensional image of the protein structure;

coloring, by the protein viewer, the three-dimensional image of the protein structure by a coloring parameter of the protein viewer; and

capturing, by the protein viewer, the plurality of two-dimensional images from different points of view after the three-dimensional image has been expanded and colored.

32. The computer-implemented method of clause 31, wherein the coloring parameter is amino acid type, and wherein the coloring of the three-dimensional image of the protein structure is by amino acid type.

33. The computer-implemented method of clause 31, wherein the coloring parameter is conservation of protein structure, and wherein the coloring of the three-dimensional image of the protein structure is by conservation of protein structure.

34. The computer-implemented method of clause 31, wherein the coloring parameter is structural quality, and wherein the coloring of the three-dimensional image of the protein structure is by structural quality.

35. The computer-implemented method of clause 31, wherein the three-dimensional image of the protein structure comprises graphical representations of atoms.

36. The computer-implemented method of clause 35, wherein the three-dimensional image of the protein structure comprises graphical representations of residues.

37. The computer-implemented method of clause 21,

wherein the plurality of convolutional neural networks and the second-layer convolutional neural network are parts of a two-dimensional image classification network, and

wherein the method further comprises training the two-dimensional image classification network by:
repeating, for different three-dimensional images of different protein structures, the following steps:
   (1) generating a different plurality of two-dimensional images of a different protein structure from a three-dimensional image of a different protein structure;
   (2) processing the different plurality of two-dimensional images using the plurality of convolutional neural networks to generate a different plurality of first-layer outputs;
   (3) combining the different plurality of first-layer outputs into a different second-layer input;
   (4) processing the different second-layer input using the second-layer convolutional neural network to generate the classification of the different protein structure; and
after each repetition of the steps (1), (2), (3) and (4), adjusting parameters of the two-dimensional image classification network according to the generated classification of the different protein structure.

38. The computer-implemented method of clause 37, wherein the adjusting of the parameters of the two-dimensional image classification network comprises adjusting parameters of the plurality of convolutional neural networks.

39. The computer-implemented method of clause 37, wherein the adjusting of the parameters of the two-dimensional image classification network comprises adjusting parameters of the second-layer convolutional neural network.

40. The computer-implemented method of clause 37, wherein the adjusting of the parameters of the two-dimensional image classification network comprises adjusting parameters of a pooling layer, and wherein the pooling layer performs the combining of the different plurality of first-layer outputs into the second-layer input.

41. A system, comprising:

a plurality of virtual cameras, configured to generate a plurality of two-dimensional images of a protein structure from a three-dimensional image of the protein structure,

wherein each camera of the plurality of virtual cameras is configured to capture a respective image of the plurality of two-dimensional images; and

a two-dimensional image classification network, configured to process the plurality of two-dimensional images to generate a classification of the protein structure.

42. The system of clause 41, wherein the protein structure comprises a target amino acid.

43. The system of clause 41, wherein the classification of the protein structure comprises a pathogenicity score.

44. The system of clause 41, wherein the two-dimensional image classification network is configured to process the plurality of two-dimensional images to determine pathogenicity of a nucleotide variant that causes a target amino acid in the protein structure to be replaced with an alternate amino acid, and wherein the classification comprises the pathogenicity of the nucleotide variant.

45. The system of clause 44, wherein the pathogenicity of the nucleotide variant is represented by a pathogenicity score.

46. The system of clause 41,

wherein the plurality of virtual cameras is configured to generate a plurality of graphical representations of amino acids in the protein structure from a three-dimensional graphical representation of the amino acids,

wherein the three-dimensional image of the protein structure comprises the three-dimensional graphical representation of the amino acids, and

wherein each image of the plurality of two-dimensional images comprises a respective graphical representation of the amino acids of the plurality of graphical representations of the amino acids.

47. The system of clause 46, wherein the amino acids comprise a target amino acid.

48. The system of clause 47, wherein the two-dimensional image classification network is configured to process the plurality of two-dimensional images to determine pathogenicity of a nucleotide variant that causes the target amino acid to be replaced with an alternate amino acid.

49. The system of clause 48, wherein the classification of the protein structure comprises a pathogenicity score.

50. The system of clause 48, wherein the plurality of virtual cameras are a part of a protein viewer, and wherein the protein viewer is configured to:

select the three-dimensional image of the protein structure;

expand the three-dimensional image of the protein structure;

color each amino acid of the plurality of graphical representations of amino acids, in the three-dimensional image of the protein structure, by amino acid type; and

capture, via the plurality of virtual cameras, the plurality of two-dimensional images from different points of view.

51. The system of clause 41, wherein the plurality of virtual cameras are a part of a protein viewer, and wherein the protein viewer is configured to:

select the three-dimensional image of the protein structure;

expand the three-dimensional image of the protein structure;

color the three-dimensional image of the protein structure by a coloring parameter of the protein viewer; and

capture, via the plurality of virtual cameras, the plurality of two-dimensional images from different points of view.

52. The system of clause 51, wherein the coloring parameter is amino acid type, and wherein the protein viewer is configured to color the three-dimensional image of the protein structure by amino acid type.

53. The system of clause 51, wherein the coloring parameter is conservation of protein structure, and wherein the protein viewer is configured to color the three-dimensional image of the protein structure by conservation of protein structure.

54. The system of clause 51, wherein the coloring parameter is structural quality, and wherein the protein viewer is configured to color the three-dimensional image of the protein structure by structural quality.

55. The system of clause 51, wherein the three-dimensional image of the protein structure comprises graphical representations of atoms.

56. The system of clause 51, wherein the three-dimensional image of the protein structure comprises graphical representations of residues.

57. The system of clause 51, wherein the three-dimensional image of the protein structure comprises graphical representations of atoms and residues.

58. The system of clause 42, further comprising a computing system, to train the two-dimensional image classification network, configured to:

repeat, for different three-dimensional images of different protein structures, the following steps:
(1) generating a different plurality of two-dimensional images of a different protein structure from a three-dimensional image of the different protein structure; and
(2) processing, by the two-dimensional image classification network, the different plurality of two-dimensional images to generate a classification of the different protein structure; and

adjust, after each repetition of the steps (1) and (2), parameters of the two-dimensional image classification network according to the generated classification of the different protein structure.

59. The system of clause 58, wherein the two-dimensional image classification network is configured to process the plurality of two-dimensional images to determine pathogenicity of a nucleotide variant that causes the target amino acid to be replaced with an alternate amino acid.

60. The system of clause 59, wherein the classification of the protein structure comprises a pathogenicity score.

61. A system, comprising:

a plurality of virtual cameras, configured to generate a plurality of two-dimensional images of a protein structure from a three-dimensional image of the protein structure,
wherein each camera of the plurality of virtual cameras is configured to capture a respective image of the plurality of two-dimensional images;

a first convolutional layer comprising a plurality of convolutional neural networks, configured to generate a plurality of first-layer outputs based on the plurality of two-dimensional images,
wherein each output of the plurality of first-layer outputs is for a respective image of the plurality of two-dimensional images, and
wherein each convolutional neural network of the plurality of convolutional neural networks is for a respective image of the plurality of two-dimensional images;

a pooling layer, configured to combine the plurality of first-layer outputs into a second-layer input; and

a second convolutional layer comprising a second-layer convolutional neural network, configured to generate a classification of the protein structure based on the second-layer input.

62. The system of clause 61, wherein the protein structure comprises a target amino acid.

63. The system of clause 61, wherein the classification of the protein structure comprises a pathogenicity score.

64. The system of clause 62, comprising a two-dimensional image classification network,

wherein the two-dimensional image classification network is configured to process the plurality of two-dimensional images to determine pathogenicity of a nucleotide variant that causes the target amino acid to be replaced with an alternate amino acid, and

wherein the two-dimensional image classification network comprises the first convolutional layer, the pooling layer, and the second convolution layer.

65. The system of clause 64, wherein the classification of the protein structure comprises a pathogenicity score.

66. The system of clause 61,

wherein the plurality of virtual cameras is configured to generate a plurality of graphical representations of amino acids in the protein structure from a three-dimensional graphical representation of the amino acids,

wherein the three-dimensional image of the protein structure comprises the three-dimensional graphical representation of the amino acids, and

wherein each image of the plurality of two-dimensional images comprises a respective graphical representation of the amino acids of the plurality of graphical representations of the amino acids.

67. The system of clause 66, wherein the amino acids comprise a target amino acid.

68. The system of clause 67, comprising a two-dimensional image classification network,

wherein the two-dimensional image classification network is configured to process the plurality of two-dimensional images to determine pathogenicity of a nucleotide variant that causes the target amino acid to be replaced with an alternate amino acid, and

wherein the two-dimensional image classification network comprises the first convolutional layer, the pooling layer, and the second convolution layer.

69. The system of clause 68, wherein the classification of the protein structure comprises a pathogenicity score.

70. The system of clause 68, wherein the plurality of virtual cameras is a part of a protein viewer, and wherein the protein viewer is configured to:

select the three-dimensional image of the protein structure;

expand the three-dimensional image of the protein structure;

color each amino acid of the plurality of graphical representations of amino acids, in the three-dimensional image of the protein structure, by amino acid type; and

capture, via the plurality of virtual cameras, the plurality of two-dimensional images from different points of view.

71. The system of clause 61, wherein the plurality of virtual cameras is a part of a protein viewer, and wherein the protein viewer is configured to:

select the three-dimensional image of the protein structure;

expand the three-dimensional image of the protein structure;

color the three-dimensional image of the protein structure by a coloring parameter of the protein viewer; and

capture, via the plurality of virtual cameras, the plurality of two-dimensional images from different points of view.

72. The system of clause 71, wherein the coloring parameter is amino acid type, and wherein the coloring of the three-dimensional image of the protein structure is by amino acid type.

73. The system of clause 71, wherein the coloring parameter is conservation of protein structure, and wherein the coloring of the three-dimensional image of the protein structure is by conservation of protein structure.

74. The system of clause 71, wherein the coloring parameter is structural quality, and wherein the coloring of the three-dimensional image of the protein structure is by structural quality.

75. The system of clause 61, wherein the three-dimensional image of the protein structure comprises graphical representations of atoms.

76. The system of clause 75, wherein the three-dimensional image of the protein structure comprises graphical representations of residues.

77. The system of clause 61, comprising:

a two-dimensional image classification network, comprising the first convolutional layer, the pooling layer, and the second convolutional layer; and

a computing system, to train the two-dimensional image classification network, configured to:

repeat, for different three-dimensional images of different protein structures, the following steps:
(1) generating, via the plurality of virtual cameras, a different plurality of two-dimensional images of a different protein structure from a three-dimensional image of a different protein structure;
(2) generating, via the plurality of convolutional neural networks, a different plurality of first-layer outputs based on the different plurality of two-dimensional images;
(3) combining, via the pooling layer, the different plurality of first-layer outputs into a different second-layer input; and
(4) generating, via the second-layer convolutional neural network, a classification of the different protein structure based on the different second-layer input; and

adjust, after each repetition of the steps (1), (2), (3) and (4), parameters of the two-dimensional image classification network according to the generated classification of the different protein structure.

78. The system of clause 77, wherein the computing system is configured to adjust parameters of the plurality of convolutional neural networks.

79. The system of clause 77, wherein the computing system is configured to adjust parameters of the two-dimensional image classification network comprises adjusting parameters of the second-layer convolutional neural network.

80. The system of clause 77, wherein the computing system is configured to adjust parameters of the pooling layer.

81. A computer-implemented method of determining pathogenicity of variants, including:

accessing a structural rendition of amino acids of a protein;

capturing a plurality of images of those parts of the structural rendition that contain a target amino acid from the amino acids; and

based at least in part on the plurality of images, determining pathogenicity of a nucleotide variant that mutates the target amino acid into an alternate amino acid.

82. The computer-implemented method of clause 81, wherein images in the plurality of images are captured from multiple perspectives.

83. The computer-implemented method of clause 82, wherein the images are captured from multiple points of view.

84. The computer-implemented method of clause 82, wherein the images are captured from multiple orientations.

85. The computer-implemented method of clause 82, wherein the images are captured from multiple positions.

86. The computer-implemented method of clause 82, wherein the images are captured from multiple zoom levels.

87. The computer-implemented method of clause 81, further comprising: activating a feature configuration of the structural rendition prior to capturing the plurality of images; and capturing the plurality of images of the parts of the structural rendition with the activated feature configuration.

88. The computer-implemented method of clause 87, wherein the feature configuration is displaying a residue detail of the amino acids.

89. The computer-implemented method of clause 87, wherein the feature configuration is displaying an atomic detail of the amino acids.

90. The computer-implemented method of clause 87, wherein the feature configuration is displaying a polymer detail of the amino acids.

91. The computer-implemented method of clause 87, wherein the feature configuration is displaying a ligand detail of the amino acids.

92. The computer-implemented method of clause 87, wherein the feature configuration is displaying a chain detail of the amino acids.

93. The computer-implemented method of clause 87, wherein the feature configuration is displaying surface effects of the amino acids.

94. The computer-implemented method of clause 93, wherein the surface effects include transparency, and color coding for electrostatic and hydrophobic values.

95. The computer-implemented method of clause 87, wherein the feature configuration is displaying density maps of the amino acids.

96. The computer-implemented method of clause 87, wherein the feature configuration is displaying supramolecular assemblies of the amino acids.

97. The computer-implemented method of clause 87, wherein the feature configuration is displaying sequence alignments of the amino acids.

98. The computer-implemented method of clause 87, wherein the feature configuration is displaying docking results of the amino acids.

99. The computer-implemented method of clause 87, wherein the feature configuration is displaying trajectories of the amino acids.

100. The computer-implemented method of clause 87, wherein the feature configuration is displaying conformational ensembles of the amino acids.

101. The computer-implemented method of clause 87, wherein the feature configuration is displaying secondary structures of the amino acids.

102. The computer-implemented method of clause 87, wherein the feature configuration is displaying tertiary structures of the amino acids.

103. The computer-implemented method of clause 87, wherein the feature configuration is displaying quaternary structures of the amino acids.

104. The computer-implemented method of clause 87, wherein the feature configuration is setting a zoom factor for displaying the structural rendition.

105. The computer-implemented method of clause 87, wherein the feature configuration is setting a lighting condition for displaying the structural rendition.

106. The computer-implemented method of clause 87, wherein the feature configuration is setting a visibility range for displaying the structural rendition.

107. The computer-implemented method of clause 87, wherein the feature configuration is color coding the structural rendition by the amino acids.

108. The computer-implemented method of clause 87, wherein the feature configuration is color coding the structural rendition by evolutionary conservations of the amino acids.

109. The computer-implemented method of clause 87, wherein the feature configuration is color coding the structural rendition by structural qualities of the amino acids.

110. The computer-implemented method of clause 81, wherein a pathogenicity determiner determines the pathogenicity of the nucleotide variant by processing, as input, the plurality of images, and generating, as output, a pathogenicity score for the alternate amino acid.

111. The computer-implemented method of clause 110, wherein the pathogenicity determiner is a neural network.

112. The computer-implemented method of clause 111, wherein the neural network is a convolutional neural network.

113. The computer-implemented method of clause 112, wherein the pathogenicity determiner processes respective images in the plurality of images through respective convolutional neural networks to generate respective feature maps for the respective images.

114. The computer-implemented method of clause 113, wherein the respective convolutional neural networks are respective instances of a same convolutional neural network.

115. The computer-implemented method of clause 114, wherein the pathogenicity determiner combines the respective feature maps into a pooled feature map and processes the pooled feature map through a final convolutional neural network to generate the pathogenicity score for the alternate amino acid.

116. The computer-implemented method of clause 113, wherein the respective images in the plurality of images are combined into a combined representation for processing by the pathogenicity determiner.

117. The computer-implemented method of clause 116, wherein the respective images are arranged as respective color channels in the combined representation.

118. The computer-implemented method of clause 116, wherein intensity values in the respective images are pixel-wise summed into pixels of the combined representation.

119. The computer-implemented method of clause 81, further including capturing respective pluralities of images for respective target amino acids that mutate to respective alternate amino acids.

120. The computer-implemented method of clause 119, further including assigning a first subset of the respective pluralities of images a benign ground truth label based on a benignness of corresponding alternate amino acids.

121. The computer-implemented method of clause 120, further including assigning a second subset of the respective pluralities of images a pathogenic ground truth label based on a pathogenicity of corresponding alternate amino acids.

122. The computer-implemented method of clause 121, further including using the respective pluralities of images with the assigned benign and pathogenic ground truth labels to train the pathogenicity determiner.

123. The computer-implemented method of clause 81, wherein the parts of the structural rendition contain the target amino acid and some additional amino acids adjacent to the target amino acid.

124. The computer-implemented method of clause 81, wherein the structural rendition is a three-dimensional (3D) structural rendition.

125. A computer-implemented method, including:

accessing image data;

modifying the image data to encode a plurality of characteristics/features of a protein; and

using the modified image data for further processing.

126. The computer-implemented method of claim 125, wherein modifying the image data includes modifying red, blue, green (R, G, B) values of pixels of the image data.

What we claim is:

1. A system comprising:
at least one processor; and
a non-transitory computer readable medium comprising instructions that, when executed by the at least one processor, cause the system to:
access a structural rendition of amino acids of a protein;
capture a plurality of images of those parts of the structural rendition that contain a target amino acid from the amino acids; and
based at least in part on the plurality of images, determine pathogenicity of a nucleotide variant that mutates the target amino acid into an alternate amino acid.

2. The system of claim 1, wherein images in the plurality of images are captured from multiple perspectives.

3. The system of claim 2, wherein the images are captured from one or more of multiple points of view, multiple orientations, multiple positions, or multiple zoom levels.

4. The system of claim 1, further comprising instructions that, when executed by the at least one processor, cause the system to:
activate a feature configuration of the structural rendition prior to capturing the plurality of images; and
capture the plurality of images of the parts of the structural rendition with the activated feature configuration.

5. The system of claim 4, wherein the feature configuration comprises displaying one or more of a residue detail of the amino acids, an atomic detail of the amino acids, a polymer detail of the amino acids, a ligand detail of the amino acids, a chain detail of the amino acids, surface effects of the amino acids, density maps of the amino acids, supramolecular assemblies of the amino acids, sequence alignments of the amino acids, docking results of the amino acids, trajectories of the amino acids, conformational ensembles of the amino acids, secondary structures of the amino acids, tertiary structures of the amino acids, or quaternary structures of the amino acids.

6. The system of claim 5, wherein the surface effects include transparency, and color coding for electrostatic and hydrophobic values.

7. The system of claim 4, wherein the feature configuration comprises:
setting one or more of a zoom factor for displaying the structural rendition, a lighting condition for displaying the structural rendition, or a visibility range for displaying the structural rendition; or
color coding the structural rendition by one or more of the amino acids, by evolutionary conservations of the amino acids, or by structural qualities of the amino acids.

8. A computer-implemented method of determining pathogenicity of variants, including:
accessing a structural rendition of amino acids of a protein;
capturing a plurality of images of those parts of the structural rendition that contain a target amino acid from the amino acids; and
based at least in part on the plurality of images, determining pathogenicity of a nucleotide variant that mutates the target amino acid into an alternate amino acid.

9. The computer-implemented method of claim 8, wherein a pathogenicity determiner determines the pathogenicity of the nucleotide variant by processing, as input, the plurality of images, and generating, as output, a pathogenicity score for the alternate amino acid.

10. The computer-implemented method of claim 9, wherein the pathogenicity determiner is a convolutional neural network or another neural network.

11. The computer-implemented method of claim 10, wherein the pathogenicity determiner processes respective images in the plurality of images through respective convolutional neural networks to generate respective feature maps for the respective images.

12. The computer-implemented method of claim 11, wherein the respective convolutional neural networks are respective instances of a same convolutional neural network.

13. The computer-implemented method of claim 12, wherein the pathogenicity determiner combines the respective feature maps into a pooled feature map, and processes the pooled feature map through a final convolutional neural network to generate the pathogenicity score for the alternate amino acid.

14. The computer-implemented method of claim 13, wherein the respective images in the plurality of images are combined into a combined representation for processing by the pathogenicity determiner.

15. The computer-implemented method of claim 14, wherein the respective images are arranged as respective color channels in the combined representation.

16. The computer-implemented method of claim 15, wherein intensity values in the respective images are pixel-wise summed into pixels of the combined representation.

17. The computer-implemented method of claim 8, wherein the parts of the structural rendition contain the target amino acid and some additional amino acids adjacent to the target amino acid.

18. The computer-implemented method of claim 8, wherein the structural rendition is a three-dimensional (3D) structural rendition.

19. A non-transitory computer-readable medium storing instructions that, when executed by at least one processor, cause a computing device to:
access a structural rendition of amino acids of a protein;
capture a plurality of images of those parts of the structural rendition that contain a target amino acid from the amino acids; and
based at least in part on the plurality of images, determine pathogenicity of a nucleotide variant that mutates the target amino acid into an alternate amino acid.

20. The non-transitory computer-readable medium of claim 19, further storing instructions that, when executed by the at least one processor, cause the computing device to determine the pathogenicity of the nucleotide variant by utilizing a neural-network-based pathogenicity determiner to process, as input, the plurality of images, and generate, as output, a pathogenicity score for the alternate amino acid.
